SEQUENCE LISTING (5 sequences)

SEQ ID NO: 1 - protein, 593 aa, Human
  PEPTIDE (1)..(593)
  ref <301>: AUTHORS: Alokail,M.S. and Peddie,M.J.
  ref <302>: TITLE: Quantitative comparison of PTH1R in breast cancer MCF7 and osteosarcoma SaOS-2 cell lines
  ref <303>: JOURNAL: Cell Biochemistry and Function
  ref <304>: VOLUME: 26
  ref <305>: ISSUE: 4
  ref <306>: PAGES: 522-533
  ref <307>: DATE: 2008-06-01
  ref <308>: DATABASE ACCESSION NUMBER: NP_000307
  ref <309>: DATABASE ENTRY DATE: 2008-08-17
  ref <313>: RELEVANT RESIDUES IN SEQ ID NO: (1)..(593)
MGTARIAPGLALLLCCPVLSSAYALVDADDVMTKEEQIFLLHRAQAQCEKRLKEVLQRPASIMESDKGWTSASTSGKPRKDKASGKLYPESEEDKEAPTGSRYRGRPCLPEWDHILCWPLGAPGEVVAVPCPDYIYDFNHKGHAYRRCDRNGSWELVPGHNRTWANYSECVKFLTNETREREVFDRLGMIYTVGYSVSLASLTVAVLILAYFRRLHCTRNYIHMHLFLSFMLRAVSIFVKDAVLYSGATLDEAERLTEEELRAIAQAPPPPATAAAGYAGCRVAVTFFLYFLATNYYWILVEGLYLHSLIFMAFFSEKKYLWGFTVFGWGLPAVFVAVWVSVRATLANTGCWDLSSGNKKWIIQVPILASIVLNFILFINIVRVLATKLRETNAGRCDTRQQYRKLLKSTLVLMPLFGVHYIVFMATPYTEVSGTLWQVQMHYEMLFNSFQGFFVAIIYCFCNGEVQAEIKKSWSRWTLALDFKRKARSGSSSYSYGPMVSHTSVTNVGPRVGLGLPLSPRLLPTATTNGHPQLPGHAKPGTPALETLETTPPAMAAPKDDGFLNGSCSGLDEEASGPERPPALLQEEWETVM

SEQ ID NO: 2 - protein, 115 aa, Human
  UNSURE (1)..(115)
  ref <301>: AUTHORS: French,D., Hamilton,L.H., Mattano,L.A. Jr., Sather,H.N.,Devidas,M., Nachman,J.B. and Relling,M.V.
  ref <302>: TITLE: A PAI-1 (SERPINE1) polymorphism predicts osteonecrosis in children with acute lymphoblastic leukemia: a report from the Children′s Oncology Group
  ref <303>: JOURNAL: Blood
  ref <304>: VOLUME: 111
  ref <305>: ISSUE: 9
  ref <306>: PAGES: 4496-4499
  ref <307>: DATE: 2008-05-01
  ref <308>: DATABASE ACCESSION NUMBER: NP_000306
  ref <309>: DATABASE ENTRY DATE: 2008-07-13
  ref <313>: RELEVANT RESIDUES IN SEQ ID NO: (1)..(115)
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 3 - protein, 177 aa, Human
  UNSURE (1)..(177)
  ref <301>: AUTHORS: Suva,L.J., Winslow,G.A., Wettenhall,R.E.H., Hammonds,R.G.,Suva,L.J., Winslow,G.A., Wettenhall,R.E.H., Hammonds,R.G.,Rodriguez,H., Chen,E.Y., Hudson,P.J., Martin,T.J. and Wood,W.I.
  ref <302>: TITLE: A parathyroid hormone-related protein implicated in malignant hypercalcemia: cloning and expression
  ref <303>: JOURNAL: Science
  ref <304>: VOLUME: 237
  ref <305>: ISSUE: 4817
  ref <306>: PAGES: 893-896
  ref <307>: DATE: 1987
  ref <308>: DATABASE ACCESSION NUMBER: P12272
  ref <309>: DATABASE ENTRY DATE: 2008-06-10
  ref <313>: RELEVANT RESIDUES IN SEQ ID NO: (1)..(177)
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 4 - DNA, 1782 bp, Human
  mRNA (1)..(1782)
  ref <301>: AUTHORS: Strausberg,R.L. et. al.
  ref <302>: TITLE: Generation and initial analysis of more than 15,000 full-length human and mouse cDNA sequences
  ref <303>: JOURNAL: Proc. Natl. Acad. Sci. U.S.A. 99 (26), 16899-16903 (2002)
  ref <304>: VOLUME: 99
  ref <305>: ISSUE: 26
  ref <306>: PAGES: 16899-16903
  ref <307>: DATE: 2002
  ref <308>: DATABASE ACCESSION NUMBER: BC110388
  ref <309>: DATABASE ENTRY DATE: 2006-08-11
atggggaccgcccggatcgcacccggcctggcgctcctgctctgctgccccgtgctcagctccgcgtacgcgctggtggatgcagatgacgtcatgactaaagaggaacagatcttcctgctgcaccgtgctcaggcccagtgcggaaaacggctcaaggaggtcctgcagaggccagccagcataatggaatcagacaagggatggacatctgcgtccacatcagggaagcccaggaaagataaggcatctgggaagctctaccctgagtctgaggaggacaaggaggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaacggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtga

SEQ ID NO: 5 - DNA, 348 bp, Human
  mRNA (1)..(348)
  ref <301>: AUTHORS: Strausberg,R.L. et. al
  ref <302>: TITLE: Generation and initial analysis of more than 15,000 full-length human and mouse cDNA sequences
  ref <303>: JOURNAL: Proc. Natl. Acad. Sci.
  ref <304>: VOLUME: 99
  ref <305>: ISSUE: 26
  ref <306>: PAGES: 16899-16903
  ref <307>: DATE: 2002
  ref <308>: DATABASE ACCESSION NUMBER: BC096144
  ref <309>: DATABASE ENTRY DATE: 2006-10-04
  ref <313>: RELEVANT RESIDUES IN SEQ ID NO: (1)..(348)
atgatacctgcaaaagacatggctaaagttatgattgtcatgttggcaatttgttttcttacaaaatcggatgggaaatctgttaagaagagatctgtgagtgaaatacagcttatgcataacctgggaaaacatctgaactcgatggagagagtagaatggctgcgtaagaagctgcaggatgtgcacaattttgttgcccttggagctcctctagctcccagagatgctggttcccagaggccccgaaaaaaggaagacaatgtcttggttgagagccatgaaaaaagtcttggagaggcagacaaagctgatgtgaatgtattaactaaagctaaatcccagtga

(19) United States
(12) Patent Application Publication
(10) Pub. No.: US 2011/0178015 A1
(43) Pub. Date: Jul. 21, 2011

(54) METHODS OF TREATMENT USING BIASED LIGANDS FOR RECEPTORS SUCH AS THE PTH RECEPTOR

(76) Inventors: Louis Luttrell, Mt. Pleasant, SC (US); Robert Lefkowitz, Durham, NC (US); Diane Gesty-Palmer, Durham, NC (US); Robert F. Spurney, Durham, NC (US)

(21) Appl. No.: 12/829,171

(22) Filed: Jul. 1, 2010

Related U.S. Application Data
(62) Division of application No. 12/060,600, filed on Apr. 1, 2008, now abandoned.
(60) Provisional application No. 60/907,439, filed on Apr. 2, 2007.

(51) Int. Cl. A61K 38/29; A61P 19/00; A61P 19/08; A61P 19/10

(52) U.S. Cl. 514/11.8

(57) ABSTRACT

Disclosed are compositions and methods for modulating the β-arrestin pathway selectively over the G protein pathway of a G protein couple receptor, such as parathyroid hormone receptor.

### CROSS-REFERENCE TO RELATED APPLICATIONS

This application is a divisional of U.S. application Ser. No. 12/060,600, filed Apr. 1, 2008, which claims the benefit of U.S. provisional application No. 60/907,439, filed Apr. 2, 2007, both of which are incorporated herein by reference in their entirety.

### STATEMENT REGARDING FEDERALLY SPONSORED RESEARCH

This invention was made with government support under RO1 DK64353 awarded by the NIH/NIDDK, and under RO1 64353, RO1 HL16037-33-37, and K12HD043446 awarded by the Arthritis Foundation Investigator Award. The government has certain rights in the invention.

### I. BACKGROUND

An emerging paradigm in seven transmembrane receptor (7TMR) biology is that both G proteins and β-arrestins can independently transduce receptor signals, and that biased ligands can selectively activate these distinct pathways. Shown herein β-arrestin biased ligands, such as, PTH-βarr, for the type I parathyroid hormone (PTH)/PTH-related protein receptor (PTH 1R), which can activate β-arrestin but not G protein signaling induces anabolic bone formation in mice, as does PTH(1-34), which activates both mechanisms. The increase in bone mineral density evoked by PTH (1-34) is attenuated in β-arrestin 2 null mice where as that to PTH-βarr is ablated. The β-arrestin 2 dependent pathway contributes primarily to trabecular bone formation and does not stimulate (markers of) bone resorption when measured. Currently employed anti-resorptive therapies aid in reducing fracture risk. However, these therapies are not sufficient to regenerate trabecular bone architecture. Thus, efforts are needed to identify anabolic agents that target osteoblast-mediated bone formation. The present methods and compositions provide in part a method of promoting bone formation, trabecular bone formation, which method can be used, for example, in the treatment of osteoporosis.

### II. SUMMARY

Disclosed are methods and compositions related to modulation of the β-arrestin pathway differentially to the G protein pathway of seven transmembrane receptors, such as the PTH1R.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1. (D-Trp12, Tyr34)-PTH(7-34) (PTH-βarr) is an Inverse Agonist for cAMP Accumulation in Primary Osteoblasts (POBs). cAMP stimulation of endogenous PTH receptor in response to PTH(1-34) and PTH-βarr was measured in POBs isolated from β-arrestin 2−/− and WT C57BL/6 mice. Cells were treated with 100 nM PTH(1-34) (PTH) or 1 μM PTH-βarr. In POBs isolated from WT and β-arrestin 2−/− mice, PTHstimulates a robust increase in cAMP. Consistent with its inverse agonist activity, PTH-βarr is unable to stimulate cAMP in WT POBs and decreases basal cAMP levels in β-arrestin 2−/− POBs. cAMP values were normalized to forskolin-induced levels. Data correspond to the mean±SEM from four independent experiments. (***, P<0.001 compared with the nonstimulated WT POB; †††, P<0.001; ††, P<0.01 compared with the non-stimulated β-arrestin 2−/− POBs).

FIG. 2 PTH-βarr stimulates β-arrestin mediated ERK1/2 activation. PTH-βarr stimulated ERK1/2 activation, was assessed in POBs isolated from β-arrestin 2−/− and WT C57BL/6 mice. POBs were treated with 100 nM PTH(1-34) (PTH) or 1 μM PTH-βarr for 5 min. WT obs treated with PTH or PTH-βarr robustly activated ERK1/2 MAP kinase. The effect of PTH-barr stimulation on ERK1/2 activation in the WT obs was absent in the β-arrestin 2−/− obs. Values presented are the fold ERK1/2 phosphorylation over non-stimulated controls. Data represent the mean±SEM from four independent experiments. (**, P<0.01 compared with the non-stimulated WT POBs; ††, P<0.01 compared with the non-stimulated β-arrestin 2−/− POBs).

FIG. 3. PTH-βarr increases lumbar spine bone mineral density. The effect of daily administration of vehicle, PTH (1-34)(PTH) or PTH-βarr on bone mineral density after 4 and 8 weeks was measured in the (a) lumbar spine of WT mice (b) lumbar spine of β-arrestin 2−/− (c) femur shaft of WT mice and (d) femoral shaft of β-arrestin 2−/− mice. These results show that the anabolic effects of PTH-Barr were in trabecular bone of the WT animals, represented by the lumbar spine, as opposed to cortical bone, found in the femur. The increase in bone mineral density seen in the PTH-βarr treated WT mice was absent in the β-arrestin 2−/− mice demonstrating that the observed anabolic effect of PTH-βarr is β-arrestin dependent. Data represents the mean±SEM of at least 7 independent mouse measurements. (*, P<0.05; **, P<0.01 compared with vehicle treated controls)

FIG. 4. β-arrestin 2 dependent signaling contributes to increases in trabecular bone but not cortical bone. Quantitative microCT of the lumbar spine was used to determine the effect vehicle, PTH (1-34) (PTH), or PTH-barr on (a) trabecular bone (Tb) density (BV/TV), (b) Tb thickness and (c) Tb number in WT and β-arrestin 2−/− mice after 8 wks of treatment. PTH and PTH-βarr increased tb density, tb thickness, and tb number in WT treated animals. The effects of PTH-βarr were absent in the β-arrestin −/− animals consistant with a b-arrestin mediated mechanism of anabolic bone formation. Data represent the mean±SEM of at least 7 independent mouse measurements. (***, P<0.001; **, P<0.01; *, P<0.05 compared with vehicle treated WT mice; if, P<0.01; t, P<0.05 compared with vehicle treated β-arrestin 2−/− mice).

FIG. 5. PTH-βarr increases serum osteocalcin and has no effect on urine Deoxypyridinoline (DPD) excretion. (a) Serum osteocalcin, a biochemical marker of bone formation was measured in WT and β-arrestin 2−/− mice after 4 weeks of treatment with vehicle, PTH (1-34) (PTH) or PTH-βarr. These results show that PTH and PTH-βarr significantly increase serum osteocalcin levels compared to placebo in WT treated mice. There was no increase in serum osteocalcin in the arrestin 2−/− mice treated with PTH-βarr compared to placebo. These results are consistent with the increases in trabecular bone formation shown in FIG. 3 and FIG. 4 and that the anabolic effects of PTH-βarr on bone are β-arrestin dependent. (b) 24 hour urine DPD, a marker of bone degradation and bone resorption, was also measured in WT and β-arrestin 2−/− mice after 4 weeks of treatment with vehicle, PTH or PTH-βarr. These results show that PTH-βarr had no significant effect on bone resorption in either WT or β-arrestin 2−/− mice compared to placebo. The increase in urine DPD excretion in the PTH treated β-arrestin 2−/− mice indicates that bone resorption can be meditated primarily through G protein dependent mechanisms. Data represent the mean±SEM of at least 7 independent mouse measurements. (***, P<0.001; *, P<0.05; compared with vehicle treated WT mice; †††, P<0.001; ††, P<0.01 compared with vehicle treated β-arrestin 2−/− mice).

FIG. 6 Distinct β-arrestin- and G protein-dependent Pathways Contribute to PTH Receptor-stimulated Gene Expression of Bone Regulatory Proteins. To determine the contributions of β-arrestin mediated signaling, to PTH receptor stimulated transcription of bone regulatory proteins, RNA was isolated from the calvaria of WT and β-arrestin 2−/− mice treated with vehicle, PTH(1-34) (PTH), or PTH-βarr. Gene expression was analyzed by quantitative RT-PCR. (a) Consistent with bone formation PTH and PTH-βarr increased osteocalcin expression in WT calvaria. In the β-arrestin −/− mice PTH induced a significant increase osteocalcin expression consistent with a G-protein mediated bone formation. In the β-arrestin −/− mice PTH-βarr decreased osteocalcin expression supporting that PTH-βarr induces osteocalcin expression through a β-arrestin dependent mechanism while additionally inhibits endogenous PTH G protein signaling. (b) and (c). PTH-βarr did not affect expression of RANKL or OPG modulators of osteoclast recruitment. Data represent the mean±SEM from six independent experiments. (***, P<0.001; **, P<0.01; *, P<0.05; compared with vehicle treated WT mice; †††, P<0.001; †, P<0.05 compared with vehicle treated β-arrestin 2−/− mice).

FIG. 7 Schematic representation of the type 1 PTH/PTHrp receptor. The predicted amino acid sequence is shown along with the predicted locations of the transmembrane domains. The large N-terminus is shown at the top of the figure. The triangle indicates the site of cleavage of the 23 amino acid signal sequence. The filled circles represent sites of N-linked glycosylation.

FIG. 8 shows a schematic of a relationship between osteoblasts and osteoclasts. As osteoblasts are activated, RANKL and OPG are produced and secreted. RANKL activates pre-osteoclasts to turn into osteoblasts. OPG inhibits RANKL. Osteoclacin is an indicator that osteoblasts have been activated and DPD is a marker showing that osteoclasts activity has been activated.

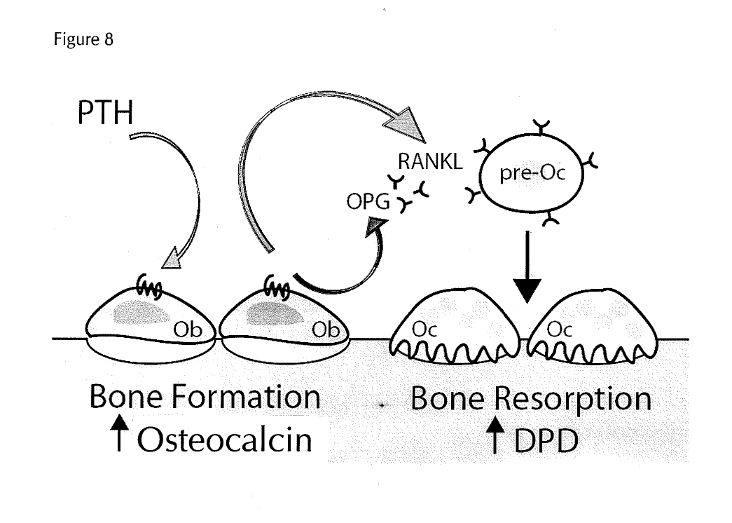

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Shown herein (D-Trp 12, Tyr34)-PTH(7-34) acts as an inverse agonist for Gs-coupling while stimulating β-arrestin-dependent activation of ERK1/2. Furthermore, the full PTH1R agonist, PTH(1-34), and the β-arrestin-selective agonist, (D-Trp12, Tyr34)-PTH(7-34), elicit distinct profiles of transcriptional activation in primary osteoblasts. In addition, in vivo, (D-Trp12, Tyr34)-PTH(7-34) treatment increases trabecular bone density in wild type, but not β-arrestin2−/− mice, indicating that activation of β-arrestin signaling pathways is sufficient to generate an anabolic response. Also, in vivo, (D-Trp12, Tyr34)-PTH(7-34) significantly increases osteoblast number, osteocalcin and OPG synthesis, without increasing osteoclast number, RANKL ligand, or bone resorption.

A. G-Protein Coupled Receptors

The G protein-coupled receptors (GPCRs) constitute the largest and most diverse superfamily of cell surface receptors in the mammalian genome. Approximately 800 distinct genes encoding functional GPCRs make up greater than 1% of the human genome (Lander, 2001; Venter, 2001). With alternative splicing, it is estimated that 1000 to 2000 discrete receptor proteins can be expressed. Such evolutionary diversity generates receptors that detect an extraordinary array of extracellular stimuli, from neurotransmitters and peptide hormones to odorants and photons of light. GPCRs function in neurotransmission, direct neuroendocrine control of physiologic homeostasis and reproduction, regulate hemodynamics and intermediary metabolism, and influence the growth, proliferation, differentiation, and death of multiple cell types. It is estimated that over half of all drugs in clinical use target GPCRs, acting either to mimic endogenous GPCR ligands, to block ligand access to the receptor, or to modulate ligand production (Flower, 1999).

Sequence similarities, hydropathy plots and a large amount of biochemical and mutagenic data support the conclusion that all GPCRs share a common seven transmembrane domain architecture. The transmembrane domains share the highest degree of sequence conservation, while the intracellular and extracellular domains exhibit extensive variability in size and complexity. The extracellular and transmembrane regions of the receptor are involved in ligand binding while the intracellular domains are important for signal transduction and for feedback modulation of receptor function. One or more sites for N-glycosylation are present within the N-terminus or, less often, the extracellular loops. Most GPCRs have in common two Cys residues that form a disulfide bridge between e1 and e2 that is critical for normal protein folding, and another Cys residue in the C terminal domain that serves as a site for palmitoylation. This lipid modification leads to the formation of a putative fourth intracellular loop.

Several classification systems have been devised that group GPCRs based upon their ligands or sequence similarities. The widely used A through F classification system of Kolakowski (Kolakowski, 1994), for example, divides the GPCRs into six families, of which three (Families A, B, and C) contain the majority of known human receptors. In this system, Family A is made up of the rhodopsin-related receptors and is by far the largest group, containing the receptors for biogenic amines and other small nonpeptide ligands, chemokines, opioids and other small peptides, protease-activated receptors, and receptors for glycoprotein hormones. Family B GPCRs, the second largest group, contains receptors that bind to higher-molecular-weight peptide hormones, such as glucagon, calcitonin and parathyroid hormone. Family C, the smallest group, contains the metabotropic glutamate receptors, the GABA_{B} receptor, and the calcium-sensing receptor.

As genome-wide data from a number of species has become available, it has been possible to model the phylogeny of the GPCRs in more detail. Analysis of the chromosomal positions and sequence fingerprints of a large number of GPCRs has led Fredriksson et al. to propose the GRAFS classification system, in which the receptors are grouped into five families: Glutamate, Rhodopsin, Adhesion, Frizzled/Taste2, and Secretin (Fredriksson, 2003). GPCRs in the GRAFS family arose from a common ancestor and evolved through gene duplication and exon shuffling. The GRAFS system contains some surprising relationships, such as the proposed link between Frizzled receptors, which are not generally thought to signal via heterotrimeric G proteins, and TAS2 group of taste receptors. Such phylogenetic linkages hint that the term ‘G protein-coupled receptor’ may be a partial misnomer for a superfamily of seven transmembrane receptors that utilize diverse signaling mechanisms.

All GPCRs function as ligand-activated guanine nucleotide exchange factors (GEFs) for heterotrimeric G proteins. The binding of a ‘first messenger’ hormone to the extracellular or transmembrane domains of the receptor triggers conformational changes that are transmitted through the intracellular receptor domains to promote coupling between the receptor and its cognate G proteins. The receptor stimulates G protein activation by catalyzing the exchange of GTP for GDP on the Gαsubunit and dissociation of the GTP-bound Gαsubunit from the Gβγsubunit heterodimer. Once dissociated, free Gα-GTP and Gβγsubunits regulate the activity of enzymatic effectors, such as adenylate cyclases, phospholipase Cβisoforms, and ion channels to generate small molecule ‘second messengers’. Second messengers, in turn, control the activity of protein kinases that regulate key enzymes involved in intermediary metabolism. Signaling continues until the intrinsic GTPase activity of the Gα subunit returns the G protein to the inactive heterotrimeric state.

### 1. GPCR Protein-Protein Interactions and GPCR Signalling

While the classical paradigm of GPCR signaling is sufficient to account for most of the rapid cellular responses to receptor activation, other protein-protein interactions account for the diversity of GPCR activity as disclosed herein. (Freedman, 1996; Hall, 2002; Brady, 2002; Maudsley, 2005; Luttrell, 2005; Luttrell, 2006; Milligan, 2001; Angers, 2002; Sexton, 2001; Foord, 1999; El Far, 2002; Bockaert, 2003). These protein-protein interactions include the formation of GPCR dimers, the interaction of GPCRs with receptor activity-modifying proteins (RAMPs), and the binding of PDZ domain containing and non-PDZ domain scaffold proteins to the intracellular loops and C-termini of receptors. These interactions modify GPCR pharmacology and trafficking, localize receptors to specific subcellular domains, limit signaling to pre-determined pathways and poise downstream effectors for efficient activation. Rather than resulting from the random collision of receptor, G protein and effector in the plane of the plasma membrane, GPCR signaling is highly pre-organized in multiprotein ‘signalsomes.’

As discussed herein two broad signaling branches flowing from a GPCR are the β-arrestin branch and the G-protein branch. Disclosed are compositions and methods that selectively activate the β-arrestin branch over the G-protein branch and the G-protein branch over the β-arrestin branch. This selective activation as shown herein results in specific biological activity and is linked to disease states and disease treatment.

### 2. β-Arrestins Function as Agonist-Regulated Scaffolds B. for GPCR Signaling

The arrestins are a family of four GPCR binding proteins that play a central role in the processes of homologous GPCR desensitization and sequestration (Luttrell, 2005; Ferguson, 2001). Two arrestin isoforms, visual arrestin (Arrestin 1; Shinohara, 1987; Yamaki, 1987) and cone arrestin (Murakami, 1993; Craft, 1994), are expressed almost exclusively in the retina and exist primarily to regulate photoreceptor function. The nonvisual arrestins, β\tilde{-}arrestin 1 (Arrestin 2; Lohse, 1990) and β-arrestin 2 (Arrestin 3; Attramandal, 1992), regulate the activity of most of the other 600 plus GPCRs in the genome. Arrestins bind tightly and specifically to GPCRs that have been phosphorylated on clusters of C-terminal Ser/Thr residues by G protein-coupled Receptor Kinases (GRKs) (Lefkowitz, 1993a) and sterically preclude further G protein activation. Not surprisingly then, it is estimated that over half of all drugs in current clinical use target GPCRs, acting either to mimic endogenous GPCR ligands, to block ligand access to the receptor, or to modulate ligand production.

Arrestin binding also controls GPCR endocytosis or sequestration. Most GPCRs undergo agonist-induced sequestration and for a majority the process involves dynamin-dependent endocytosis via clathrin-coated pits (Zhang, 1996). The two β-arrestins, but not the visual arrestins, contain LIEF/L and RxR motifs in the C-terminal regulatory domain that engage clathrin and the β 2 adaptin subunit of the AP-2 complex, respectively, leading to the clustering of receptors in clathrin-coated pits (Krupnick, 1997; Laporte, 1999). Once internalized, GPCR-arrestin complexes are targeted to early endosomes, in which they are sorted either for resensitization and recycling to the plasma membrane or targeted for degradation. The longevity of the receptor-β-arrestin interaction is a major determinant of the fate of internalized receptors, with receptors that dissociate from β-arrestin upon endocytosis tending to undergo rapid recycling, while receptors that form stable receptor-β-arrestin complexes are slowly recycled or targeted to lysosomes and degraded (Oakley, 1999).

Unlike the catalytic GPCR-G protein interaction, β-arrestins bind GPCRs in a stable bimolecular complex, wherein they function as adapters, physically linking the receptor to the endocytic machinery. The arrestin bound receptor is in a high agonist affinity state, analogous the classical GPCR-G protein ‘ternary complex’ (Gurevich, 1999; Hoist, 2001), which has prompted some authors to describe the receptor-arrestin complex as an ‘alternative ternary complex’ (Gurevich, 1999). It was the discovery that this complex is itself a GPCR signal transducer that has led to the hypothesis that β-arrestins serve as adapters not only in the context of GPCR sequestration, but also in linking activated GPCRs to cellular signaling systems (Luttrell, 2005a; Luttrell, 2005b; Miller, 2001; Perry, 2002a; Luttrell, 2002a; Shenoy, 2003; Shenoy, 2005a; Shenoy, 2005b; Shenoy, 2005c 8). A number of catalytically-active proteins have been shown to bind β-arrestins and undergo O-arrestin-dependent recruitment to agonist-occupied GPCRs; among them Src family tyrosine kinases (Luttrell, 1999a; DeFea, 2000a; Barlic, 2000), components of the extracellular signal-regulated kinase 1 and 2 (ERK1/2) and c-Jun N-terminal Kinase 3 mitogen-activated protein (MAP) kinase cascades (McDonald, 2000; DeFea, 2000b; Luttrell, 2001; Tohgo, 2002; Tohgo, 2003; Wel, 2003; Caunt, 2006; Gesty-Palmer, 2006; Jafri, 2006), the E3 ubiquitin ligase, Mdm2 (Shenoy, 2001), and the cAMP phosphodiesterases, PDE4D3/5 (Perry, 2002b).

Note that while some signaling proteins, e.g. ERK1/2, apparently bind to both β-arrestin isoforms, others, e.g. JNK3, bind selectively, creating the possibility of isoform-selective signal transduction.

### 1. GPCR Bound by Agonist Activates Two Signal Pathways

Agonist-binding to a GPCR simultaneously initiates two antagonistic processes; heterotrimeric G protein activation leading to G protein dependent signal production, and receptor desensitization leading to attenuated receptor-G protein coupling and waning signal intensity over time (Freedman, 1996; Luttrell, 2005a). Since β-arrestin binding uncouples receptor and G protein, the transmission of G protein-dependent and β-arrestin-dependent signals are mutually exclusive, at least at the level of the individual receptor.

Disclosed herein is that β-arrestin-dependent formation of a multi-protein signalsome complex leads to the initiation of a distinct second path of GPCR signaling that is initiated as the receptor undergoes desensitization and enters the endocytic pathway. Indeed, comparisons of the time course of ERK1/2 activation resulting from heterotrimeric. G protein activation and from the β-arrestin-dependent formation of an ERK1/2 activation complex on the angiotensin AT1a, lysophosphatidic acid (LPA), type 1 parathyroid hormone (PTH) and β2-adrenergic receptors (β2-AR) demonstrate that the onset of β-arrestin dependent ERK1/2 activation coincides with the waning of G protein signaling and persists as receptors internalize (Luttrell, 2001; Ahn, 2004; Azzi, 2003; Gesty-Palmer, 2005; Shenoy, 2006).

### 2. GPCRs Employ Several Mechanisms to Regulate ERK1/2 Activity

The ability of GPCRs to activate the ERK1/2 MAP kinase cascade is central to their regulation of cell proliferation, differentiation and chemotactic migration (van Biesen, 1996; Gutkind, 1998; Luttrell, 1999b; Luttrell, 2002b). MAP kinases are regulated via a series of parallel kinase cascades, each composed of three kinases that successively phosphorylate and activate the downstream component. In the ERK1/2 cascade, for example, the proximal kinases, cRaf-1 and B-Raf, phosphorylate and activate MEK1 and MEK2. MEK 1 and 2 are dual function threonine/tyrosine kinases that, in turn, carry out the phosphorylation and activation of ERK1/2. (Pearson, 2001). It is now clear that multiple signals contribute to GPCR-stimulated ERK1/2 activation. These include classical second messenger-dependent pathways, e.g. Gs-, adenylyl cyclase-, and PKA- and EPAC dependent activation of the small G protein Rap1 (Vossler, 1997; Grewal, 2000); protein kinase C-dependent activation of c-Raf1 (Hawes, 1995); and calcium and cell adhesion-dependent activation of the focal adhesion kinase, Pyk2 (Lev, 1995; Dikic, 1996). GPCRs can also trigger Ras-dependent ERK1/2 activation by ‘transactivating’ receptor tyrosine kinases such as the EGF (Daub, 1997; Prenzel, 1999) and Platelet-Derived Growth Factor (PDGF) receptors (Heeneman, 2000; Linseman, 1995). In addition, several GPCRs, including the protease-activated receptor PAR2, AT1AR, β2AR, PTH1R, and the neurokinin NK-1, and vasopressin V2 receptors, have been shown to activate ERK1/2 using receptor-bound β-arrestins as ligand regulated scaffolds (DeFea, 2000b; Luttrell, 2000; Tohgo, 2002; Tohgo, 2003; Wei, 2003; Caunt, 2006; Gesty-Palmer, 2006; Jafri, 2006). Both O-arrestin isoforms form a complex with the component kinases of the ERK1/2 cascade, and appear to act as ligand regulated scaffolds in a manner functionally analogous to the S. cervisiae scaffold protein, STE5p (Elion, 2001), with which they share no sequence homology. Given this diversity, it is not surprising that in most cells types, GPCRs can employ two or more mechanisms to activate ERK1/2, or that the dominant mechanism(s) vary with receptor and cell type. What is, perhaps, surprising, is that the function of ERK1/2 appears to be dictated by the mechanism of activation, with some signals promoting nuclear translocation and others cytosolic retention of ERK1/2.

C. Parathyroid Hormone

PTH (parathyroid hormone) is a major regulator of calcium and phosphate homeostasis, while parathyroid hormone-related peptide (PTHrP) has important developmental roles. Both peptides signal through the same receptor, the PTH/PTHrP receptor, i.e. type 1 parathyroid hormone receptor. It is known to directly stimulate osteoblast mediated bone formation, and indirectly stimulate bone resorption by upregulating the production of soluble factors, such as RANKL, that promote osteoclast differentiation and function. As a result, the net effect of PTH administration on bone metabolism is determined by the relative activation of these two opposing processes. With continuous exposure, bone resorption exceeds new bone formation, resulting in osteomalacia, whereas intermittent exposure stimulates net bone formation. Despite the limitations imposed by osteoblast-osteoclast coupling, intermittent administration of the PTH agonist peptide, PTH(1-34) forms the basis of current anabolic therapy for the treatment of severe osteoporosis.

PTH is a circulating hormone comprised of 84 amino acids. It is produced in the parathyroid glands and acts primarily on bone and kidney to maintain extracellular calcium levels within normal limits. PTH is secreted from the chief cells of the parathyroid glands primarily in response to low extracellular calcium, but also in response to elevated extracellular phosphate. PTH is a true hormone in that it is produced by a gland and then travels through the bloodstream to act at its target tissues. The N-terminal 34 amino acids of PTH and PTHrP are sufficient for efficient activation of the PTH/PTHrP receptor. In the kidney, PTH reduces calcium excretion by increasing calcium reabsorption in the distal convoluted tubule. It furthermore prevents phosphate reabsorption primarily by affecting the expression levels of two different sodium-phosphate co-transporters, NPT-2a and NPT-2c, both of which are localized in the brush border membrane of the proximal tubules. In bone, PTH effects are equally complex and lead to a net release of calcium and phosphate from the matrix into the blood. (See Gensure R C, Gardella T J, Jüppner H. Parathyroid hormone and parathyroid hormone-related peptide, and their receptors. Biochem Biophys Res Commun. 328:666-678, 2005.).

Examplary sequences of PTH1R ligands are shown in SEQ ID NOs: 2-3.

1. Parathyroid Hormone Analogues

Structure-activity relationships for parathyroid hormone have been extensively investigated using a variety of peptide fragments and/or modified parathyroid fragment analogs (Potts, 2005). Since the phenomenon of β-arrestin-dependent signal transduction was not recognized at the time the majority of this work was performed, most PTH-derived peptides have been characterized only as agonists or antagonists for Gs-dependent cAMP generation or Gq/11-dependent phosphatidylinositol production.

At least two PTH fragments, hPTH(1-34) and (Leu27)cycloGlu22-Lys26hPTH(1-31)NH2 have been developed for the treatment of osteoporosis. One of these, recombinant (r)hPTH(1-34), is FDA approved for the treatment of severe osteoporosis and is marketed under the trade name of Forteo. (Leu27)cycloGlu22-Lys26hPTH(1-31)NH2 is in phase II clinical trials under the trade name Ostabolin-C. In addition, the native hormone hPTH(1-84) has also completed clinical trials (Whitfield, 2006). All three of these peptides stimulate bone growth, reinforce bone microstructure weakened by estrogen deprivation and reduce further fracturing, but hPTH(1-34) and hPTH(1-84) are not β-arrestin biased specific ligands as discussed herein, but (Leu27)cycloGlu22-Lys26hPTH(1-31)NH₂ has not been tested to show whether it is a biased ligand.

In terms of biased agonism (Biased ligand) with respect to the property of selective engagement of G protein- or β-arrestin-signaling, the parathyroid hormone analog (D-Trp¹², Tyr³⁴) PTH(7-34) acts as an inverse agonist for PTH1 receptor-Gs coupling, while promoting arrestin-dependent sequestration (Gardella, 1996; Sneddon, 2004). Trp^{l}-PTHrP(1-36) possesses the opposite activity profile promoting Gs-coupling and cAMP production without inducing β-arrestin recruitment or desensitization (Bisello, 2002). The β-arrestin-selective biased agonist, (D-Trp¹², Tyr³⁴) PTH(7-34), has been shown in vitro to elicit β-arrestin-dependent ERK1/2 activation while functioning as an inverse agonist (inhibitor) of PTH1R-mediated cAMP production (Gesty-Palmer, 2006).

D. Parathyroid Hormone Receptor Type 1 (PTH1R)

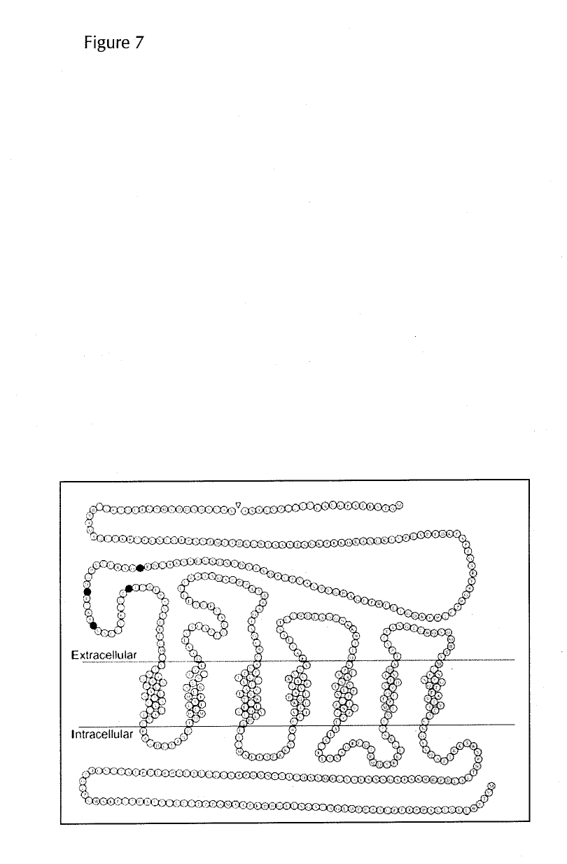

PTH and PTHrP act through a common receptor, the PTH/PTHrP receptor, which is a class B G-protein-coupled receptor (FIG. 7). This family of receptors includes the receptors for secretin, vasoactive intestinal peptide, glucagon, glucagon-like peptide, corticotrophin-releasing factor, growth hormone-releasing hormone, pituitary adenylate cyclase-activating peptide, gastric inhibitory peptide, calcitonin, and a few other peptide hormones.

A second receptor that binds PTH in vitro, the PTH2 receptor, is most closely related to the PTH/PTHrP receptor (51% amino acid identity). PTH acts as an agonist at the human PTH2 receptor, but shows little or no agonism at the rat or fish homologs of this receptor. PTHrP shows no agonism at any of the known PTH2 receptors. The lack of response to PTH by the rat PTH2 receptor, and the predominant localization of this receptor to the hypothalamus, suggest physiological roles distinct from the regulation of calcium homeostasis. Indeed, further investigation led to the discovery of TIP39, a 39 amino acid peptide structurally related to PTH and PTHrP, which appears to be the natural ligand for this receptor. Postulated biological activities for TIP39 and the PTH2 receptor include nociception and possibly the regulation of pituitary hormone secretion. (See Gensure R C, Gardella T J, Jüppner H. Parathyroid hormone and parathyroid hormone-related peptide, and their receptors. Biochem Biophys Res Commun. 328:666-678, 2005.).

PTH activity is mediated through the type I PTH/PTH-related peptide receptor (PTH1R), a seven-transmembrane receptor (7TMR) highly expressed in the kidney and bone. The intracellular signaling pathways activated by the PTH 1R receptor include G_{S} mediated adenylate cyclase-cAMP-PKA and G_{q/11}-mediated PLCβ-inositol 1,4,5-trisphosphate (IP₃)-PKC signaling pathways. Additionally, PTH activates the Raf-MEK-ERK MAP kinase (MAPK) cascade through both PKA and PKC in a cell-specific and G protein-dependent manner.

Disclosed herein, β-arrestins, in addition to playing a negative regulation effect on G-protein signaling, also act as signal transducers through the formation of scaffolding complexes with accessory effector molecules such as Src, Ras, raf, ERK1/2, JNK3, and MAPK kinase 4 (MKK4), and JNK3. PTH stimulation of PTH1R promotes translocation of both β-arrestin 1 and β-arrestin 2 to the plasma membrane, association of the receptor with β-arrestins, the internalization of the receptor/β-arrestin complexes and activation of ERK1/2. Disclosed herein are compositions that cause the β-arrestin activation pathway of a GPCR to be activated more than the G-protein pathway.

E. GPCR related diseases

1. Bone Disorders

Bone disorders can be treated by using a β-arrestin biased ligand as discussed herein. For example, Osteoporosis due to aging (senile osteoporosis); hypogonadism (post menopausal in women or hypoandrogenic in men); endogenous or exogenous corticosteroid excess (chronic prednisone administration) could all be treated using biased ligands.

Fracture repair (traumatic fractures) or implant anchorage (bone grafting) can be treated or enhanced using the biased ligands disclosed herein. For example, by administering the biased ligands as disclosed herein to a subject having a bone fracture or having an implant that has been placed such that the implant is anchoring to the bone, the subject's fracture can heal faster and the implant can anchor quicker than without the administration of the biased ligand or a control.

Osteoporosis is a significant clinical health threat. In the U.S., approximately 10 million individuals are estimated to have the disease and almost 34 million more have low bone mass, placing them at increased risk for developing osteoporosis.

Osteoporosis results largely from a net imbalance between osteoblast-mediated bone formation and osteoclast-mediated bone resorption. This imbalance results in low bone mass and microarchitectural deterioration which leads to bone fragility, susceptibility to fractures, as well as increased morbidity and mortality. Associated medical costs exceed 18 billion dollars per year.

The actions of PTH on bone, however, are complex. PTH is known to have both anabolic as well as catabolic effects on bone. Despite the data supporting the importance of PTH-mediated signals in bone remodeling, little is known about the mechanistic basis for these effects.

2. GPCR Related Diseases and Biased Ligands

GPCR related diseases that can be treated with the disclosed biased ligands include pulmonary and cardiovascular disease, allergies/allergic diseases, immunological diseases, psychiatric disorders, psychological disorders, dermatological diseases, neurological diseases, autonomic diseases, inflammatory diseases, endocrine or metabolic diseases (e.g., diabetes and obesity), genitourinary disorders, and opthamological diseases (e.g. glaucoma).

### a) G Protein-Selective Biased Agonists

Drugs that activate G but recruit β-arrestin less than a control, could be advantageous in a setting where sustained GPCR activity without desensitization is desirable. Examples would include bronchial asthma (long-acting β2-adrenergic receptor agonist to promote bronchodilation); allergic rhinitis (α1-adrenergic receptor agonist that relieves nasal congestion without causing rebound nasal congestion). Inotropic drugs for short term parenteral use in the treatment of cardiogenic or septic shock, e.g. α-adrenergic receptor agonists that did not cause tachyphylaxis, could be superior to current agents.

3. PTH1R has Two Distinct Signaling Paths

G protein- and β-arrestin-dependent signaling are two distinct and pharmacologically separable mechanisms. It has been shown that stimulation of the PTH1R activates ERK1/2 MAP kinase by two temporally distinct mechanisms, one G protein-dependent pathway and the other β-arrestin-dependent, and that these two mechanisms of PTH1R signaling (G protein versus β-arrestin) can be selectively stimulated through the use of PTH analogues that discriminate between the G-protein-coupled and β-arrestin coupled conformations of the receptor.

β-arrestin 2 has been shown to influence bone remodeling and the anabolic effects of intermittent PTH(1-34) administration in murine models. Ferrari et al. reported that intermittent administration of PTH(1-34) fails to increase bone mineral content and trabecular bone volume in β-arrestin2^{-/-} mice. This effect was attributed to the loss of classic β-arrestin desensitization of G protein coupled signaling, increased and sustained cAMP. Disclosed herein are β-arrestin pathway biased ligands that elicit bone formation and methods of utilizing these biased ligands.

F. Ligands

### 1. Agonist, Antagonist, Inverse Agonist, Biased Ligand, Biased Agonist

### a) The Ternary Complex Model of GPCR Function

GPCRs transmit signals intracellularly by functioning as ligand-activated guanine nucleotide exchange factors (GEFs) for heterotrimeric G proteins. G protein activation is initiated through hormone-driven changes in the tertiary structure of the transmembrane heptahelical receptor core that are transmitted to the intracellular transmembrane loops and carboxyl terminus. These conformational changes alter the ability of the receptor to interact with intracellular G proteins and catalyze the exchange of GDP for GTP on the heterotrimeric G protein alpha subunit. The GTP-bound alpha subunit stimulates its cognate downstream effectors, e.g. an adenylate cyclase or phospholipase C, conveying information about the presence of an extracellular stimulus to the intracellular environment.

Previous work involving a large number of GPCRs, has affirmed the hypothesis that the receptor exists in spontaneous equilibrium between two conformations (active: R*; inactive: R) that differ in their ability to activate G proteins (Samama et al., 1993). In the native state the receptor is maintained predominantly in the R conformation by intramolecular interactions within the transmembrane helical bundle, i.e. the spontaneous equilibrium heavily favors the inactive R state. Agonist binding, or selective mutagenesis, relieves these constraints, allowing the receptor to ‘relax’ into the R* conformation that enables G-protein coupling. The extended ternary complex model developed to explain these phenomena proposes that the intrinsic efficacy of a ligand is a reflection of its ability to alter the equilibrium between R and R* (Lefkowitz et al., 1993).

### b) Three State to Multi-State Models

While the ternary complex model can sufficiently explain the properties of agonism, antagonism, partial agonism, and inverse agonism, it is still limited in that it accommodates the existence of only two functional receptor states. In a two state model, i.e. where only a single R* conformation exists, the agonist pharmacology of a receptor should be the same regardless of the response being measured. Yet a paradoxical reversal of relative efficacy of agonists has been described for several GPCRs that activate more than one stimulus-response element, including the 5-HT2c receptor (Berg et al., 1998), pituitary adenylate cyclase-activating polypeptide (PACAP) receptor (Spengler et al., 1993), dopamine D2 receptor (Meller et al., 1992), and neurokinin NK-1 receptor (Sagan et al., 1999). Although differential stimulus pathway activation can occur through a strength of signal type of mechanism, i.e. a highly efficacious agonist might activate two pathways whereas a weaker agonist may activate only the more sensitive one, the reversal of the relative efficacy of different agonists acting on the same receptor cannot be explained on the basis of a two state model.

The demonstration that GPCRs exhibit ligand-specific activation states led to the proposal that two or more active states of the same receptor may exist. In these three-state or multistate models, agonists are predicted to induce distinct “active” conformations of the receptor by differentially exposing regions of the intracellular domains involved in coupling to different G protein pools. Indeed, multiple G protein-coupled states of the α₂-adrenergic receptor can be distinguished using a variety of guanine nucleotide analogues (Seifert et al., 1999). Similarly, several receptor mutations have been described that produce constitutive activity that is restricted to a single signaling pathway among those ordinarily activated by the receptor (Perez et al., 1996). These mutations presumably restrict conformational isomerization of the receptor to a certain subset that promotes specific G protein coupling conformations. While the behavior of a mutated receptor cannot be extrapolated a priori to its wild type counterpart, these data clearly demonstrate that subtle changes in receptor structure outside of the G protein-coupling domains, as might occur upon binding different agonist ligands, can alter G protein selectivity (Kenakin, 2002).

Biophysical evidence also supports the concept that different GPCR ligands induce distinct populations of receptor microconformation (Ghanouni et al., 2001). Fluorescence lifetime spectroscopy of β2 adrenergic receptors fluorescently labeled at Cys265 reveals a Gaussian distribution of environments for the probe reflecting continuous fluctuations in receptor conformation. Addition of agonist or antagonist ligands changes the distribution of receptor conformations, reflecting the stabilization of a specific subset of conformations. Moreover, different agonists select different arrays of receptor conformation, consistent with the induction of ligand-selective active states.

The existence of multiple active receptor conformations makes it plausible that agonists can change not only the degree, but also the ‘quality’ of receptor activation. It is known that different areas of the cytosolic loops on receptors activate different G-proteins (Wade et al., 1999). It is thus predictable that agonists producing distinct tertiary conformations of a receptor could expose these different G-protein-activating sequences so as to produce differential, or ‘biased’, activation of G proteins. This multi-state model of GPCR activation provides the theoretical basis for the concept of signaling-selective agonism, also referred to as ‘agonist-specific trafficking of receptor signaling’ (Kenakin, 1995b; Kenakin, 1995c).

Thus, GPCRs exist in a spontaneous equilibrium between states that do not activate downstream signaling and states that do activate down stream signaling, through a variety of paths, such as the G protein path and the β arrestin path. Furthermore, since there are multiple signaling paths there are more than one equilibria that when altered can cause a downstream signaling event. See Maudsley, S., Martin, B. and Luttrell, L. M. Perspectives in Pharmacology: The origins of diversity and specificity in G protein-coupled receptor signaling. J. Pharm. Exp. Therapeutics. 314:485-494, 2005.

Definitions that relate to the conformational state are as follows. An agonist is a ligand that binds to a receptor, such as a GPCR, and stabilizes one or more receptor conformations that promote an increase in signaling activity relative to the unliganded (unbound) state. A ligand interacts with all or part of the receptor structure that is involved in binding the naturally-occurring compound(s) that regulate receptor activity in vivo. This word does not encompass allosteric modulators, which are compounds that interact with regions of the receptor outside the ligand binding pocket, but that change receptor structure in such a manner as to alter its response to a ligand.

An antagonist is a ligand that binds to a receptor, such as a GPCR, without measurably affecting the spontaneous equilibrium of the receptor between its active and inactive state(s). It has no measurable effect on the spontaneous equilibrium of receptor conformations relative to the unliganded state. Its presence can be detected only when a ligand that does alter the conformational equilibrium is simultaneously present, since the antagonist will compete for binding and lower the potency of the ‘activating’ ligand. A neutral antagonist will reduce the potency of an ‘inverse agonist’ just as it will that of an agonist.

An inverse agonist is a ligand that binds to a GPCR and stabilizes the inactive conformation of the receptor, causing a reduction in the basal signaling activity of the receptor relative to the unliganded state. Under conditions of low basal activity, an inverse agonist cannot be distinguished from an antagonist using conventional measures of signaling efficacy.

A biased ligand is any ligand that acts either as an agonist, antagonist, or inverse agonist for less than all of the possible down stream signaling activities of a receptor.

A biased agonist is a biased ligand that binds to a receptor, such as a GPCR, and stabilizes a subset of the possible active conformations of the receptor, generating only part of the full response profile relative to the unliganded state. Embodied in the concept of multiple active states that reflect different receptor conformations, a biased agonist will exhibit different agonist, antagonist or inverse agonist properties, depending on the signaling output being measured.

A biased ligand will produce true ‘reversal of efficacy’, meaning that its characterization as an agonist, antagonist or inverse agonist will be different, depending on the signaling output being measured. For example, (D-Trp¹²,Tyr³⁴)-PTH(7-34), a biased agonist for the type 1 PTH receptor, behaves as an inverse agonist with respect to activation of cAMP production (lowers basal activity relative to the unliganded state), while behaving as an agonist with respect to activation of arrestin-dependent receptor internalization or signaling (increases receptor internalization and ERK1/2 activity relative to the unliganded state).

G. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Control is used herein. In certain embodiments, a control can be a reference ligand, such as an agonist, antagonist, inversed agonist, or biased ligand. By reference ligand is meant any ligand having a known activity profile for a particular receptor, such as a GPCR. In certain embodiments a control refers to any comparative state, for example, an activated state vs a control state which would be an unactivated state. For example, a control can be non-stimulated in a specific assay of cAMP production or ERK1/2 phosphorylation. Alternatively, a control can be a comparison performed under conditions where a downstream element in a signaling pathway has been genetically deleted, such as performing ERK1/2 phosphorylation assay under conditions where β-arrestin expression has been down regulated. A control is well understood in the art and where not specifically recited it can be understood by the context with which it is being used.

Anabolic bone formation is bone formation that is an increase in the rate of new bone formation in excess of bone resorption that causes a net increase in bone mass. It is anabolic in that it is distinguished from the pure antiresorptive approach of increasing bone mass, which does not stimulate bone formation but slows the rate of breakdown.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

H. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular biased ligand is disclosed and discussed and a number of modifications that can be made to a number of molecules including the biased ligands are discussed, specifically contemplated is each and every combination and permutation of biased ligands and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those indicated by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example, PTH as well as any other proteins or peptides disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

### b) Sequences

There are a variety of sequences related to, for example, PTH1R as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

### c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

### d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of PTH1R or the genomic DNA of PTH1R or they can interact with the polypeptide PTH1R. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of PTH1R aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

4. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A.R. Gennaro, Mack Publishing Company, Easton, Pa. 1995.

5. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

### a) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

6. Peptides

### a) Protein Variants

As discussed herein there are numerous variants of the PTH1R protein that are known and herein contemplated. In addition, to the known functional PTH1R strain variants there are derivatives of the PTH1R proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions

| | Exemplary Conservative Substitutions, |
|---|---|
| Original Residue | others are known in the art. |
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:1 sets forth a particular sequence of PTH1R. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—CH═CH—(cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron.

Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with PTH1R such that PTH1R activates the β-arrestin pathway over the G protein pathway as discussed herein. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that can be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes can be immunized in vitro, e.g., using the cells containing the 7tmrs, such as PTH1R as described herein.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti PTH1R antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa.

Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The disclosed compositions and methods can also be used for example as tools to isolate and test new drug candidates for a variety of GPCR related diseases.

9. Compositions Identified by Screening with Disclosed Compositions/Combinatorial Chemistry

### a) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed in SEQ ID NO:1 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, PTH1R, are also disclosed. Thus, the products produced using the combinatorial or screening approaches that involve the disclosed compositions, such as, PTH1R, are also considered herein disclosed.

It is understood that the disclosed methods for identifying molecules that inhibit the interactions between, for example, PTH1R and PTH can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dye. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example an extracellular portion of PTH1R is attached to a DNA binding domain of a transcriptional activation protein, such as Gal 4. By performing the Two-hybrid technique on this type of system, molecules that bind the extracellular portion of PTH1R can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

### b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, PTH1R, are also disclosed. Thus, the products produced using the molecular modeling approaches that involve the disclosed compositions, such as, PTH1R, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

10. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

I. Methods of Making

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:3, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tent-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

J. Methods

Disclosed are methods of promoting anabolic bone growth. The methods comprise administering to a patient in need thereof a biased agonist for the PTH1 receptor that can stimulate β-arrestin-mediated signaling independent of G protein-mediated signaling. The biased agonist is administered in an amount sufficient to effect promotion of bone growth.

Therapeutics previously demonstrated to generate anabolic bone growth through stimulation of the PTH 1 receptor include agonists such as PTH (1-34) and PTH (1-84). In contrast to the biased agonists disclosed herein, the prior agonists bind the PTH 1 receptor and stimulate G protein-mediated activation of adenylate cyclase and inositol-1,4,5-trisphosphate (IP₃) production (Dunlay et al, Am. J. Physiol. Renal Physiol. 285(2):F223-231 (1990); Guo et al, Endocrinology 136(9):3884-3891 (1995)).

Biased agonists for the PTH1 receptor suitable for use in the instant invention have signaling properties that result in anabolic bone formation, including generation of trabecular bone architecture. A biased agonist disclosed herein is [D-Trp(12), Tyr(34)]bPTH(7-34)amide (PTH-IA), is an inverse agonist for the PTH1 receptor (Goldman et al, Endocrinology 123(5):2597-2599 (1988); U.S. Pat. No. 4,968,669; Bachem).

The pharmacologic action of PTH-IA has been demonstrated in vitro to be mediated by β-arrestins, not through G protein-mediated mechanisms (Gesty-Palmer et al, J. Biol. Chem. 281:10856 (2006)). The in vivo effects of administration of PTH-IA on anabolic bone formation in mice have also been studied and the results demonstrate that PTH-IA can stimulate trabecular bone formation through a G protein-independent, β-arrestin-dependent mechanism. (See Examples that follow.) Further, PTH-1A appears to uncouple the anabolic effects of PTH1 receptor stimulation from PTH1 receptor stimulated bone resorption. Available data suggest that PTH-stimulated bone resorption may be a G protein dependent process. Biased agonists disclosed herein, such as PTH-IA, which specifically stimulate β-arrestin mediated bone formation, can be expected to offer a significantly improved biologic specificity and safety profile for treatment of metabolic bone disease.

Also disclosed are derivatives of PTH-IA and, in addition, other biased agonists of the PTH1 receptor, can also be used in the present method of promoting bone growth. Examples include human PTH(7-34), [Leu(11)-D-Trp(12)]hPTHrP(7-34)-amide, [D-Trp(12)]bPTH(7-34)-amide, and [Bpa(2), Ile(5), Trp(230, Tyr(36)]PTHrP-(1-36)-amide. Also disclosed are methods of identifying other suitable biased agonists (e.g., other PTH analogues that are inverse agonists of the PTH 1 receptor). Methods of identifying suitable β-arrestin biased ligands include fluorescence resonance energy transfer (FRET)- and bioluminescent resonance energy transfer (BRET)-based assays to assess β-arrestin recruitment and stimulating efficacy. Other methods include receptor/β-arrestin co-immunoprecipitation, receptor/β-arrestin crosslinking, receptor/β-arrestin biomolecular fragmentation complementation, receptor/β-arrestin translocation imaging, receptor internalization, receptor phosphorylation, and β-arrestin associated phosphorylation of mitogen activated protein (MAP) kinases.

Also disclosed are compositions comprising the biased agonists formulated with an appropriate carrier. Formulation techniques known in the art can be used, for example, as described in Remington's Pharmaceutical Sciences, 17th ed., Mack Publishing Company, Easton, Pa., (1985). The composition can be present, for example, as a solution (e.g., a sterile solution) or suspension. The composition can be present dosage unit form (e.g., as a tablet or capsule). The nature of the formulation can vary depending, for example, on the agonist and on the route of administration.

Representative delivery regimens include, without limitation, oral, parenteral (including subcutaneous, transcutaneous, intramuscular and intravenous), rectal, buccal (including sublingual), transdermal, and intranasal. While the biased agonists of the invention, like the currently FDA approved PTH(1-34) peptide, can be administered by injection (e.g., subcutaneous injection (see http://pi.lilly.com/us/forteo-pi.pdf)), intranasal administration of an appropriately formulated biased agonist may be preferred.

In general, compositions, such as the biased agonists, or salts thereof, can be administered in amounts between about 0.01 and 10 μg/kg body weight per day, preferably, from about 0.05 to about 2.5 μg/kg body weight per day. For a 70 kg human female, the daily dose of PTH-IA, for example, can range from about 3.5 μg/kg to about 175 μg/kg, preferably from about 5 μg/kg to about 150 μg/kg. Dosages can be delivered by a single administration, by multiple applications, or via controlled release, as needed to achieve the results sought.

Optimum dosing regimens can be readily determined by one skilled in the art and can vary with the biased agonist, the patient and the effect sought.

The disclosed biased agonists can be used in the prevention and treatment of a variety of mammalian conditions characterized by loss of bone mass. For example, the biased agonists can be used for the prophylaxis and therapeutic treatment of osteoporosis and osteopenia. It can also be used in the therapeutic treatment of hyperparathyroidism and its associated bone diseases, as well as forms chondrodysplasia, and hypercalcemia. The methods disclosed herein can be used in treating humans and non-human mammals (e.g., horses and cattle).

See also Gesty-Palmer et al, J. Biol. Chem. 281(16):10856 (2006), as well as U.S. Pat. No. 7,169,567, U.S. Pat. No. 7,153,951 U.S. Pat. No. 7,150,974, U.S. Pat. No. 7,022,851, U.S. Pat. No. 4,968,669 and US Pub. Appln. 20060229240 (including but not limited to the disclosures in these patent documents of formulation/administration details and therapeutic applications).

Methods of Screening for Biased Ligands

There are variety of assays which can be used for determining activation of a GPCR. For example, two pathways can be assayed for activation.

G protein activity can be assayed by determining the level of calcium, cAMP, diacylglycerol, or inositol triphosphate in the presence and absence of the ligand (or candidate ligand). G protein activity can also be assayed, for example, by determining phosphatidylinositol turnover, GTP-γ-S loading, adenylate cyclase activity, GTP hydrolysis, etc. in the presence and absence of the ligand (or candidate ligand). (See, for example, Kostenis, Curr. Pharm. Res. 12(14): 1703-1715 (2006).

For β-arrestin activation, β-arrestin recruitment to the GPCR or GPCR internalization can be assayed in the presence and absence of the ligand (or candidate ligand). Advantageously, the β-arrestin function in the presence and absence of a ligand (or candidate ligand) is measured using by resonance energy transfer, bimolecular fluorescence, enzyme complementation, visual translocation, co-immunoprecipitation, cell fractionation or interaction of β-arrestin with a naturally occurring binding partner. (See, for example, Violin et al, Trends Pharmacol. Sci. 28(8):416-427 (2007); Carter et al, J. Pharm. Exp. Ther. 2:839-848 (2005).)

GRK activity can be used as a surrogate for β-arrestin function, β-arrestin function mediated by a GPCR in response to a ligand (or candidate ligand) can thus be reflected by changes in GRK activity, as evidenced by changes in receptor internalization or phosphorylation.

The relative efficacies for G protein activity and β-arrestin functions for a given ligand, such as a biased ligand, or candidate ligand, acting on a GPCR can be determined by assays in eukaryotic cells (e.g., mammalian cells (e.g., human cells), insect cells, avian cells, or amphibian cells, advantageously, mammalian cells). Appropriate assays can also be performed in prokaryotic cells, reconstituted membranes, and using purified proteins in vitro. Examples of such assays include, but are not limited to, in vitro phosphorylation of purified receptor by GRXs, GTP-γ-S loading in purified membranes from cells or tissues, and in vitro binding of purified β-arrestins to purified receptors upon addition of ligand (or candidate ligand) (with or without GRXs present in the reaction). (See, for example, Pitcher et al, Science 257:1264-1267 (1992); Zamah et al, J. Biol. Chem. 277:31249-31256 (2002); Benovic et al, Proc. Natl. Acad. Sci. 84:8879-8882 (1987).

In certain embodiments, an assay for G protein activation and an assay for β-arrestin can be performed, and then, for example, the relative activities of G protein and β-arrestins activation can be compared. From this a type of biased ligand can be determined. This situation can be compared as fold activity with a comparison of the various fold activities. For example, relative to a control a ligand could have 0.5 times the activity for a G protein pathway and could have 1.5 times the activity for a β-arrestins pathway. This ligand could then be classified as having a 3× β-arrestins biased ligand relative to a G protein pathway. It is clear from this example, that relative activities can be obtained for individual pathways relative to a control and that the activities of two or more pathways can be compared to characterize a biased ligand. It is understood that ligands having at least or less than or equal to, as well as less than or equal to, greater than or equal to 0.001, 0.002, 0.003, 0.004, 0.005, 0.006, 0.007, 0.008, 0.009, 0.01, 0.02, 0.03, 0.04, 0.05, 0.05, 0.06, 0.06, 0.07, 0.08, 0.09, 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 4.0, 5.0, 6.0, 7.0, 8.0, 9.0, 10.0, 20.0, 30.0, 40.0, 50.0, 60.0, 70.0, 80.0, 90.0, 100.0, 200.0, 300.0, 400.0, 500.0, 600.0, 700.0, 800.0, 900.0, or 1000.0.

Disclosed are methods of identifying biased ligands of a GPCR, such as the PTH receptor. Such methods can comprise: i) determining the effect of a test compound on GPCR-mediated G-protein activity, and ii) determining the effect of the test compound on GPCR-mediated β-arrestin function, wherein a test compound that has a greater positive effect on GPCR-mediated β-arrestin function than on GPCR-mediated G-protein activity, relative to a reference agonist for both GPCR-mediated G-protein activity and GPCR-mediated β-arrestin function, is a biased ligand. Such methods can be used to identify a candidate therapeutic that can be used to modulate (e.g., stimulate (enhance) or inhibit) a physiological process.

For example, candidate therapeutics can be identified by: i) determining the effect of a test compound on G-protein activity mediated by a GPCR relevant to the physiological process, and ii) determining the effect of the test compound on β-arrestin function mediated by that GPCR, wherein a test compound that has a greater positive effect on β-arrestin function than on G-protein activity mediated by the GPCR, relative to a reference agonist for both the G-protein activity and β-arrestin function mediated by the GPCR, is such a candidate therapeutic. Included in this aspect of the invention are methods of identifying agents suitable for use in treating cardiovascular diseases/disorders (including hypertension, heart failure, coronary artery disease, pulmonary hypertension, peripheral vascular disease or arrhythmia), as well as pulmonary diseases/disorders (such as asthma, chronic obstructive airway disease and pulmonary fibrosis), ophthalmologic diseases/disorders (such as glaucoma), hematologic diseases/disorders (including thrombolytic disorders), endocrine or metabolic diseases/disorders (e.g., diabetes and obesity), neurological or psychiatric diseases/disorders (including Parkinsonism or Alzheimer's), as well as other diseases/disorders including those referenced below.

A fluorescence resonance energy transfer (FRET)-based assay can be used to assess β-arrestin/G protein pathway activation. β-arrestin/G protein pathway activation can be measured as the rate of β-arrestin recruitment to a receptor in response to ligand, where the receptor/β-arrestin interaction is measured by FRET or bioluminescent resonance energy transfer (BRET). For example, β₂AR-mCFP and β-arrestin-mYFP undergo FRET after addition of agonists with a quantifiable rate. This rate of FRET increase is a measure of ligand-stimulated GRK activity, which regulates β-arrestin function, and thus quantifies a ligand's β-arrestin/GRK efficacy. This method can be adapted for use with a fluorescence plate reader for high-throughput screening of agonists and antagonists, which can thereby provide a rapid screen for β-arrestin/GRK biased ligands. β-arrestin/GRK function can be measured for all manner of 7TMRs, e.g., the PTH type 1 receptor.

Other assays that can be used to measure β-arrestin function include: receptor/β-arrestin co-immunoprecipitation, receptor/β-arrestin crosslinking, receptor/β-arrestin BRET, receptor/β-arrestin bimolecular fragmentation complementation, receptor/β-arrestin translocation imaging, receptor internalization, receptor phosphorylation, and β-arrestin associated phosphorylated ERK (Violin et al, Trends Pharmacol. Sci. 28(8):416-422 (2007)). As described above, approaches that can be used to measure G-protein mediated signaling function include assays for adenylate cyclase and/or cyclic AMP accumulation (ICUE (DiPilato et al, Proc. Natl. Acad. Sci. USA 101:16513 (2004)), radioimmunoassays, ELISAs, GTPase assays, GTPgammaS loading assays, intracellular calcium accumulation assays, phosphotidyl inositol hydrolysis assays, diacyl glycerol production assays (e.g., liquid chromatography, FRET based DAGR assay (Violin et al, J. Biol. Chem. 161:899 (2003)), receptor-G protein FRET assays, measures of receptor conformation change, receptor/G protein co-immunoprecipitation, ERK activation, phospholipase D activation, ion channel activation (including electrophysiologic methods), and cyclic GMP changes. (See, for example, Thomsen et al, Curr. Opin. Biotech. 16:655-665 (2005).)

Depending on the assay, any assay, that is chosen, such as cAMP production, you can rank order any set of ligands. For example, one can test 100 compositions or compounds for cAMP activation from the PTH1R and then rank those compositions or compounds from 1-100 based on their ability to activate the cAMP pathway relative to a control. This process can be repeated for a different assay(s), for example, recruitment of arrestins, and this produces a different ranking. In this way one can produce a profile for a given compound or composition which represents the compound or composition's ability to function in a variety of assays.

In certain embodiments, molecules are chosen that are β-arrestin agonists but are an antagonist or inverse agonist for G-protein activation, meaning produces less cAMP formation and/or calcium flux assay across the membrane but produces increased ERK1/2 activation and/or recruitment of β-arrestin to receptor.

In certain embodiments, the mutant PTH1R receptor, H23RPTHR, a naturally occurring mutation so that the receptor having a mutation of histidine and arginine at position 23 is used because it is partially activated at the basal level, and inverse agonism can be seen.

Bone density and bone mass can be measured. Quantitative measure of the amount of calcium hydroxy-apatite per unit volume of bone can be done by Dual Energy X-ray Absorbtion (DEXA). DEXA is a method where X-rays are taken, typically of the of the lumbar spine, hip or forearm, with X-rays of two different energies. The tissue penetration of these two different X-rays are compared, and the ratio provides a two dimensional projection of bone mineral across a three dimensional area. Bone density can also be determined by high resolution CT scan, which also provides micro-architectural information, such as bone volume and number and thickness of trabeculae or circumference and thickness of cortical bone.

Trabecular bone is composed of a spongy network of bony plates that occupies the marrow cavity of cancellous bone, providing weight-bearing strength with minimal weight. Cortical bone is the dense outer layer of bone that provides strength to the weight-bearing limbs. In osteoporosis, there is a loss of trabecular bone resulting in fewer and thinner trabeculae, decreased compressive strength and resiliency, and an increased propensity to fracture, most notably in the lumbar vertebrae, pelvis and femoral neck. Bone microarchitecture, e.g. bone volume, trabecular number, trabecular thickness, cortical circumference and cortical thickness can be measured by high resolution CT scan.

Bone formation and turnover can be estimated in the clinical setting by measuring markers of osteoblastic bone formation and osteoclastic bone degradation in samples of blood and urine. Bone formation rates are measured by assaying markers of osteoblast activity such as osteocalcin, bone alkaline phosphatase, procollagen 1C- and N-terminal propeptides. Bone degradation rates are assessed by measuring markers of osteoclast activity, such as deoxypiridoline crosslinks (DPD), collagen 1C and N-terminal telopeptides. These measures are often used clinically as surrogate markers of response to therapy.

Disclosed are methods of modulating a seven transmembrane receptor, comprising contacting a seven transmebrane receptor with a biased ligand.

Also disclosed are methods wherein the biased ligand can selectively activate the 3-arrestin pathway of the seven transmembrane receptor.

Also disclosed are methods wherein the seven transmembrane receptor comprises the parathyroid hormone (PTH)/PTH-related protein receptor (effects of PTH 1R).

Also disclosed are methods wherein the parathyroid hormone (PTH)/PTH-related protein receptor (PTH1R) is a type I receptor.

Also disclosed are methods wherein the PTH1R activation produces an increase in OPG and a decrease in RANKL.

Also disclosed are methods wherein the PTH1R activation does not cause an increase in DPD production.

Also disclosed are methods wherein the β-arrestin pathway of the seven transmembrane receptor is activated more than the G-protein pathway of the seven transmebrane receptor.

Also disclosed are methods wherein the biased ligand induces anabolic bone formation.

Also disclosed are methods wherein the biased ligand increases bone mineral density in an organism.

Also disclosed are methods wherein the biased ligand increases trabecular bone formation.

Also disclosed are methods wherein the biased ligand increases osteoblast activity relative to a control while at a similar time does not increase osteoclast activity.

Also disclosed are methods wherein the biased ligand does not couple osteoblast and osteoclast activity.

Also disclosed are methods wherein the biased ligand increases osteoblastic bone formation markers without increasing production of markers of increasing osteoclast formation.

Also disclosed are methods wherein the biased ligand does not increase osteoclast recruitment relative to a control.

Also disclosed are methods wherein the biased ligand does not increase osteoclast differentiation relative to a control.

Also disclosed are methods wherein the biased ligand comprises (D-Trp12, Tyr34)-PTH(7-34).

Also disclosed are methods wherein the biased ligand increases ERK1/2 activation while not increasing heterotrimeric G protein activation relative to PTH.

Also disclosed are methods wherein the biased ligand increases MAP kinase activation while not increasing heterotrimeric G protein activation relative to PTH.

Also disclosed are methods further comprising the step of identifying a subject in need of modulation of a seven transmebrane receptor.

Also disclosed are methods wherein the subject has a bone disorder.

Also disclosed are methods wherein the bone disorder is osteoporosis.

Also disclosed are methods wherein the modulation of the seven transmebrane receptor is monitored by the step of analyzing a biofluid of the subject for markers indicating biased ligand modulation.

Also disclosed are methods wherein the biofluid is urine.

Also disclosed are methods wherein the biofluid is serum.

Also disclosed are methods wherein the marker is osteocalcin.

Also disclosed are methods wherein the marker is increased relative to a control.

Also disclosed are methods wherein the marker is deoxypyridinoline (DPD).

Also disclosed are methods wherein the marker is not increased relative to a control comprising activation using a non-biased ligand.

Also disclosed are methods wherein the non-biased ligand comprises PTH.

Disclosed are methods of analyzing activity of a composition comprising, a) contacting the composition with a GPCR, b) determining the activation of a first signal transduction pathway of the GPCR, producing a first activation result, c) determining the activation of a second signal transduction pathway of the GPCR, producing a second activation result, and wherein the first activation result and the second activation result produce an activity profile of the composition.

Also disclosed are methods wherein the GPCR is PTH1R.

Also disclosed are methods wherein the first signal transduction pathway is the G protein pathway.

Also disclosed are methods wherein the step of determining activation of the first signal transduction pathway comprises assaying cAMP activation.

Also disclosed are methods wherein the second signal transduction pathway is the β-arrestin pathway.

Also disclosed are methods wherein the step of determining the activation of the second signal transduction pathway comprises assaying β-arrestin recruitment.

Also disclosed are methods wherein the step of determining the activation of the second signal transduction pathway comprises assaying ERK1/2 activation.

Also disclosed are methods wherein method further comprises d) contacting the GPCR with a control e) determining the activation of a first signal transduction pathway of the GPCR, producing a first activation control result, f) determining the activation of a second signal transduction pathway of the GPCR, producing a second activation control result, and wherein the first activation control result and the second activation control result produce an activity profile of the composition.

Also disclosed are methods further comprising the step of comparing the first activation result with the first activation control result.

Also disclosed are methods further comprising the step of comparing the second activation result with the second activation control result.

Also disclosed are methods further comprising the step of selecting a composition based on a desired activation profile.

Also disclosed are methods wherein the desired activation profile comprises activation of a β-arrestin pathway with reduced activation of the G protein pathway.

Also disclosed are methods wherein a subject is treated with the disclosed compositions. Also disclosed are methods, wherein a subject has been diagnosed as needing a treatment for one or more of the disorders disclosed herein, and/or is tested for the disorder prior to or as part of the treatment process.

### K. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Results

(1) (D-Trp12, Tyr34)-PTH(7-34) (PTH-βarr), Stimulates β-arrestin mediated ERK1/2 activation, independent of G protein signaling, in osteoblasts.

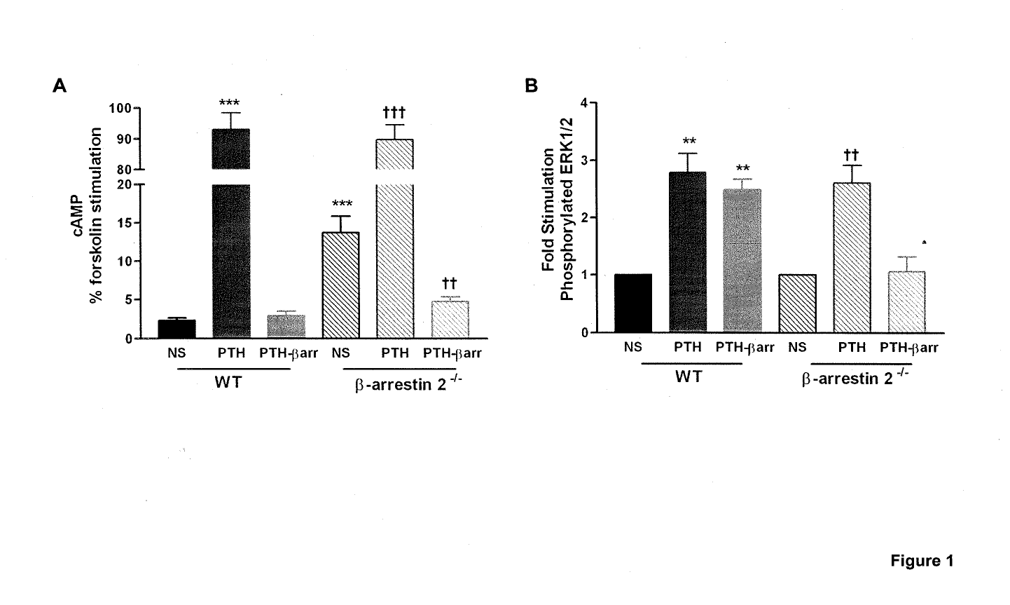

To test whether PTH-βarr exhibits a biased response under native conditions, cAMP accumulation in response to PTH(1-34) and PTH-βarr stimulation of endogenous PTH1R in primary murine osteoblasts (POB) (FIG. 1a) was examined. In confluent POB cultures isolated from WT and β-arrestin 2−/− C57BL/6 mice, the basal cAMP levels in the β-arrestin 2−/− POB were significantly higher compared to WT POB. The increased basal cAMP in the β-arrestin cells is likely due to the loss of β-arrestin mediated desensitization of PTH1R and/or other Gs coupled 7TMRs.

Treatment of both WT and β-arrestin 2−/− cells with 100 nM PTH(1-34) for 5 min generated robust increases in cAMP. There was no significant difference in cAMP generated between WT and β-arrestin 2−/−POB at 5 min. Consistent with inverse agonist activity, treatment of WT POB with 1 μM PTH-βarr did not increase cAMP while treatment of the β-arrestin 24-POB significantly decreased the elevated basal cAMP levels (FIG. 1A). Stimulation of POB cultures with PTH(1-34) or PTH-βarr did not activate Gq/11 PI hydrolysis (data not shown).

PTH(1-34) and PTH-βarr stimulated ERK1/2 MAP kinase activation was assessed in WT and β-arrestin 2−/− POB after treatment for 5 min with 100 nM PTH(1-34) or 1 μM PTH-βarr (FIG. 1b). In WT POB, both agents increased ERK1/2 phosphorylation approximately 3 fold over basal. β-arrestin 2−/− POB responded to PTH(1-34) much as WT POB, indicating that the full agonist peptide can activate ERK1/2 through classical G protein-dependent pathways in the absence of β-arrestin2. In contrast, PTH-βarr failed to activate ERK1/2 in β-arrestin 2−/− POB (FIG. 1B), demonstrating that ERK1/2 activation by PTH-Barr in WT POB is β-arrestin mediated and independent of G protein signaling.

(2) Intermittent Activation of the β-Arrestin Pathway Increases Bone Density In Vivo.

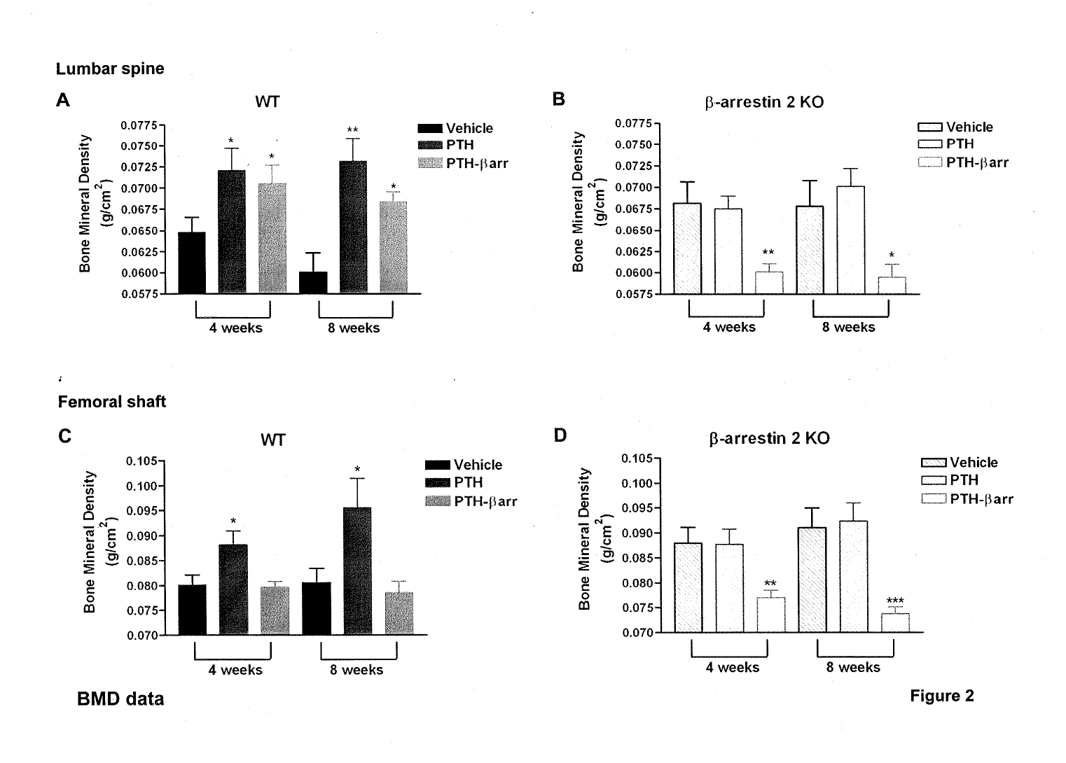

β-arrestin 2−/− mice are fertile and present no gross phenotypic abnormalities. Further, no gross alterations in skeletal morphology or size were detected by x-ray analysis of β-arrestin 2−/− mice compared to 6 WT mice (data not shown). To examine the contribution of β-arrestin mediated signaling to regulation of the anabolic effects of PTH on bone, 9 week old WT and β-arrestin 2−/− mice were treated with intermittent (i.e. once daily) IP injection of PTH (1-34) (40 mg/kg/day), the β-arrestin biased agonist PTH-βarr (40 mg/kg/day). Its usually mg/kg/day) or vehicle. Bone mineral density (BMD) measurements were obtained at baseline and serially over 4 to 8 wks (FIG. 2). At baseline, 9 wk old β-arrestin 2−/− mice had significantly lower 1-spine BMD compared to 9 week-old WT mice (WT 0.0678 g/cm₂±0.0008; β-arrestin 2−/−0.0648 g/cm₂±0.0009; p=0.012). There were no significant differences in whole body BMD or femoral BMD between the WT or β-arrestin 2−/− mice. As expected, at 4 and 8 weeks WT mice treated with PTH(1-34) showed marked increases in their lumbar spine and femoral BMD compared to vehicle treated mice (FIGS. 2A and C). Consistent with earlier reports, these increases in BMD were absent in the PTH(1-34) treated β-arrestin 2−/− mice (FIGS. 2B and D). WT mice treated with PTH-βarr (40 mg/kg/day), a β-arrestin biased agonist and inhibitor of G protein signaling, also showed significant increases in BMD in the lumbar spine (FIG. 2A). Treatment with PTH-βarr did not significantly affect femoral BMD in WT animals (FIG. 2C). Administration of PTH-βarr to β-arrestin 2−/− mice resulted in decreases in both lumbar spine and femoral BMD (FIG. 2D). Since PTH-βarr generates a subset of PTH1R signals in WT, but not β-arrestin −/− cells, independent of heterotrimeric G protein activation, these data are consistent with PTH-βarr induced changes in bone metabolism that are transmitted by PTH1R receptor ‘coupling’ to β-arrestin. The decrease in BMD in β-arrestin 2−/− mice treated with PTH-βarr, are likely due to the inhibition of G protein mediated signaling as well as the absence of β-arrestin 2 mediated signaling. These results, taken together, indicate that PTH1R stimulated anabolic effects in trabecular bone have discrete β-arrestin mediated and G protein mediated components.

(3) Contribution of Pth1R Stimulated β-Arrestin Mediated Signaling to Trabecular Bone Mass and Microarchitecture.

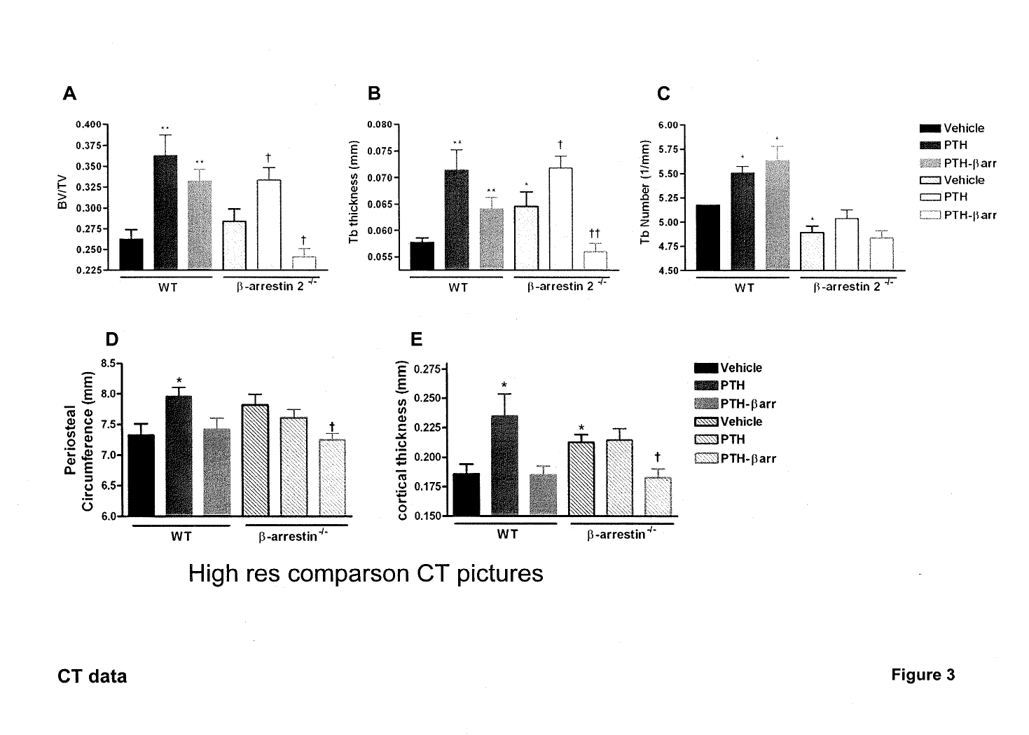

Quantitative microCT (qCT) measurements of the lumbar spine were acquired from WT and β-arrestin 2−/− mice after 8 weeks of treatment with vehicle, PTH(1-34), or PTH-βarr. There was no significant difference in the overall trabecular bone density (BV/TV) between vehicle treated WT and β-arrestin 2−/− mice (FIG. 3A). However, with respect to trabecular microarchitecture, after 8 weeks of treatment with vehicle, the β-arrestin 2−/− mice had significantly greater trabecular thickness compared to WT mice (FIG. 3B) and significantly lower trabecular number compared to WT mice (FIG. 3c). These differences in trabecular bone architecture in sham treated animals reflect two potential contributing processes 1) the loss of β-arrestin mediated signaling and 2) the exaggeration of Gs signaling due to the loss of β-arrestin desensitization.

Micro qCT analysis of lumbar vertebrae showed WT mice treated with daily administration of PTH (1-34) for 8 weeks had significantly increased lumbar spine trabecular bone density compared to vehicle treated animals (FIG. 3A). Specifically, PTH(1-34) induced significant increases in trabecular thickness (FIG. 3B) and trabecular number (FIG. 3B) After 8 weeks, PTH-βarr, a biased agonist that inhibits G protein mediated signaling while activating β-arrestin mediated signaling, induced a significant increase lumbar spine trabecular bone density in WT mice compared to vehicle treated animals (FIG. 3A). Further, PTH-βarr also induced significant increases in trabecular thickness (FIG. 3B) and trabecular number (FIG. 3C) in WT mice.

To test whether the anabolic effects of PTH-βarr on trabecular bone formation required the activation of a β-arrestin mediated signaling mechanism, β-arrestin 2−/− mice were also treated with PTH(1-34) and PTH-βarr. β-arrestin 2−/− mice treated with PTH(1-34) demonstrated a net increase trabecular bone density compared to vehicle treated β-arrestin mice. However the percent increase in Tb bone density in the PTH(1-34) treated β-arrestin mice (17%) was less than that in the WT treated mice (38%) (FIG. 3A). β-arrestin 2−/− mice treated with PTH(1-34) had significant increases in trabecular thickness (FIG. 3B) but not trebecular number (FIG. 3C) compared to vehicle treated β-arrestin 2−/− mice. PTH (1-34) is known to induce both Gs/cAMP and β-arrestin dependent signals. Thus the effects of PTH(1-34) stimulation on trabecular micro architecture of the β-arrestin 24-mice can be attributed to the loss of PTH (1-34) stimulated and/or excessive Gs signaling.

The anabolic effects of PTH-βarr in the WT animals were lost in PTH-βarr treated β-arrestin 2−/− mice. Compared to vehicle treated β-arrestin 2−/− mice, PTH-βarr treated mice exhibited significant decreases in trabecular bone volume (FIG. 3A) and trabecular thickness (FIG. 3B). The increase in trabecular number seen in the WT mice treated with PTH βarr was also absent in the β-arrestin 2−/− mice (FIG. 3c). The absence of an anabolic effect in the β-arrestin 2−/− mice indicates that the effects of PTH-βarr are β-arrestin dependent. Further, the decrease in trabecular bone density and trabecular thickness can be explained by both the loss of β-arrestin dependent signaling in the knockout animals in combination with the inhibition of endogenous PTH stimulated G protein dependent signaling events by PTH-βarr.

Finally, the effects of PTH(1-34) and PTH-βarr on cortical bone were examined by qCT of the midfemoral shaft (FIGS. 3D and E). Comparison of vehicle treated animals after 8 weeks, showed no difference in periosteal circumference between the WT and β-arrestin 2−/− mice. However the β-arrestin 2−/− mice had greater midshaft cortical thickness than vehicle treated WT mice. After 8 wks of PTH(1-34) WT mice showed increased femoral periosteal circumference and increased cortical thickness. The biased agonist, PTH-βarr had no effect on these cortical indices in WT mice. In the β-arrestin 24-mice, PTH (1-34) had no significant effect periosteal circumference or cortical thickness while PTH-βarr significantly decreased periosteal circumference and cortical thickness. There were no significant effects of PTH(1-34) or PTH-βarr on WT or β-arrestin−/− endosteal bone surfaces (data not shown).

(4) Alterations Histomorphometric Indices Induced by β-Arrestin 2-Mediated Signaling

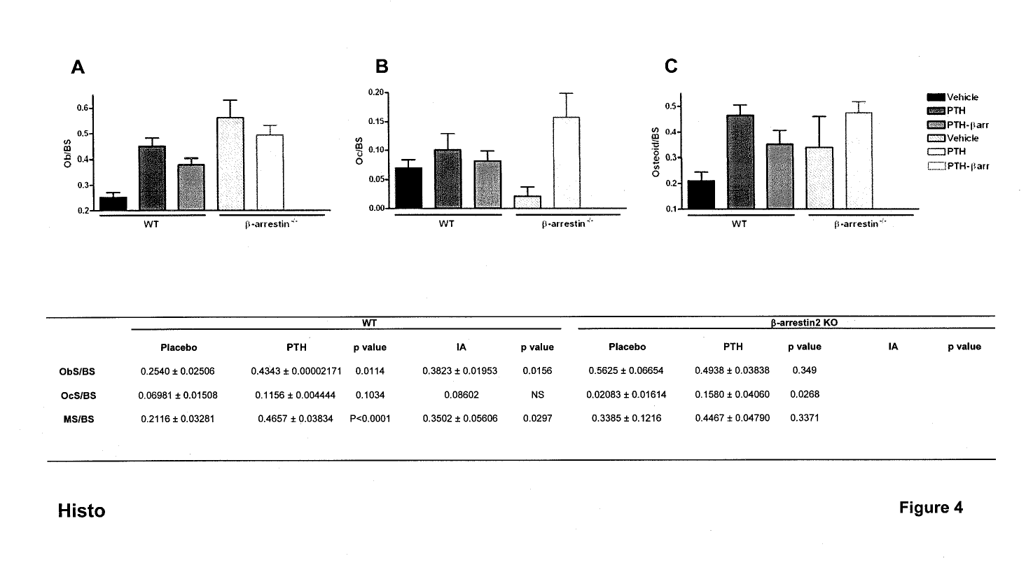

Dynamic histomorphometric data were consistent with the qCT of trabecular bone morphology. After 8 wks, vehicle treated β-arrestin 24-mice had greater osteoblast surface than vehicle treated WT (FIG. 4A) but the osteoclast surface and osteoid surface were not significantly different in these two groups (FIGS. 4B and C). Consistent with anabolic bone formation produced by selective activation of β-arrestin mediated signaling quantitative histomorphometric analyses of lumbar spine sections show that WT mice treated with either PTH(1-34) or PTH-βarr had increased osteoblast surface (FIG. 4A) and osteoid (FIG. 4C) compared to their vehicle treated counterparts. As expected there was an increase in osteoclast surface in the PTH (1-34) treated animals. Interestingly PTH-βarr treatment had no effect on osteoclast recruitment. The finding that PTH-βarr increased osteoblastic activity in WT mice whereas PTH(1-34), but not PTH-barr, accelerates osteoclast formation in the absence of O-arrestin 2, indicates that β-arrestin dependent signaling can be sufficient to stimulate osteoblastic bone formation but that osteoblast-osteoclast coupling requires G protein activation.

(5) Effects of β-arrestin mediated signaling on serum and urine markers of bone metabolism.

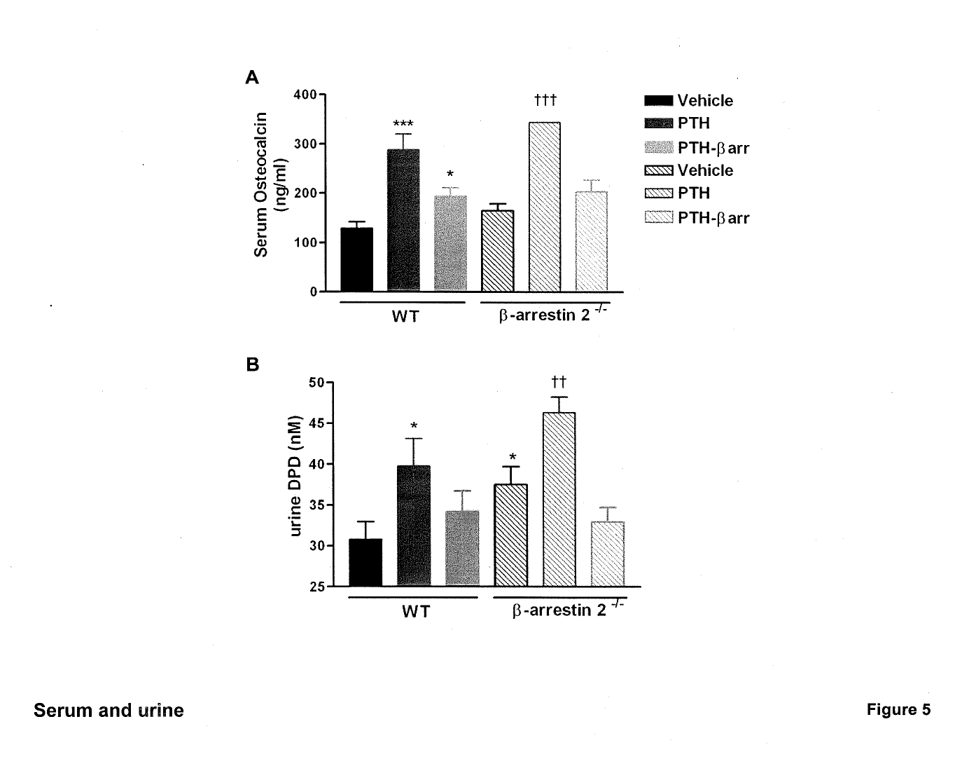

To delineate the cellular mechanisms contributing to the metabolic effects of PTH(1-34) and PTH-βarr administration in WT and β-arrestin 2−/− mice, serum and urine markers of bone turnover were assessed. Basal serum osteocalcin, a biochemical marker of bone formation, was not significantly different between WT and β-arrestin 2−/− (WT, 184.0±9.038; β-arrestin 2−/−, 210.6±11.36; p=0.068). Osteocalcin was significantly increased in WT mice treated with either PTH(1-34) or PTH-βarr compared to vehicle treated mice (FIG. 5A). Serum osteocalcin was also increased in the β-arrestin 2−/− mice treated with PTH(1-34) compared to vehicle. However, there was no significant change in serum osteocalcin in the PTH-βarr treated β-arrestin 2−/− mice, further supporting the idea that the anabolic effects of PTH-Barr on bone are β-arrestin dependent.

265. 24 hour urine deoxypyridinoline (DPD), a marker of bone degradation and bone resorption, was also measured. Vehicle treated β-arrestin 2−/− mice had significantly higher urine DPD than vehicle treated WT counterparts, consistent with greater baseline osteoclast activity in the absence of b-arrestin2. Urine DPD was significantly increased in both WT and β-arrestin 2−/− mice treated with PTH(1-34) compared to vehicle treated animals (FIG. 5B). PTH-βarr however had no significant effect on urine DPD markers of bone resorption in WT or β-arrestin 2−/− mice compared to vehicle. The increase in urine DPD excretion in the PTH(1-34) treated β-arrestin 2−/− mice compared to WT further supports the hypothesis that osteoblast-osteoclast coupling is meditated primarily through G protein dependent mechanisms that are disinhibited in the absence of β-arrestin 2.

(6) Distinct β-Arrestin- and G Protein-Dependent Pathways Contribute to PTH Receptor Stimulated Expression of Bone Regulatory Protein Genes.

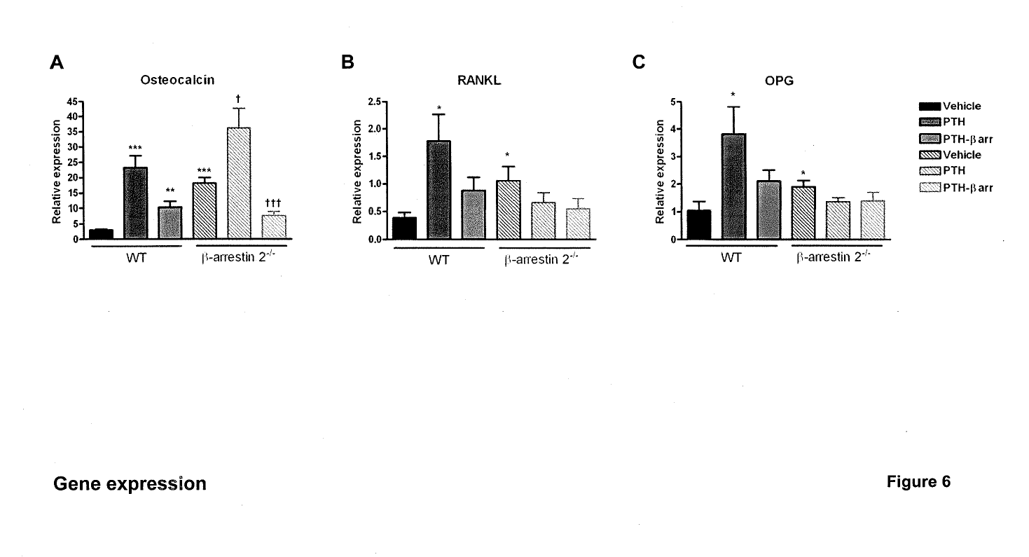

To determine the contribution of β-arrestin mediated signaling to PTH1R-stimulated transcription of bone regulatory proteins, calvarial RNA was isolated from WT and β-arrestin 2−/− mice treated with PTH(1-34), PTH-βarr or vehicle. Gene expression for osteocalcin, as well as receptor activator of nuclear factor-KB ligand (RANKL) and osteoprotegrin (OPG), which activate and inhibit osteoclastic bone resorption respectively, was analyzed by quantitative RTPCR (FIG. 6 A-C).

In vehicle treated animals, the expression of osteocalcin mRNA was higher in the β-arrestin 2−/− mice compared to WT mice consistent with the histomorphometric results showing significantly higher Ob/Bs in the β-arrestin 2−/− mice compared to WT. Both PTH(1-34) and PTH-βarr induced increases in expression of osteocalcin mRNA in WT treated animals compared to their vehicle treated counterparts (FIG. 6A) as expected with bone formation. PTH treatment also significantly increased the osteocalcin expression in the β-arrestin 2−/− mice, while PTH-βarr induced a decrease in expression of osteocalcin.

As for modulators of osteoclast activity, the expression of RANKL and OPG mRNA was higher in the vehicle treated β-arrestin 2−/− mice compared to WT mice. The increase in RANKL mRNA abundance was consistent with the significantly higher urine DPD observed in vehicle treated β-arrestin 2−/− mice compared to WT. Only PTH(1-34) induced increases in in vivo expression of RANKL and OPG in WT treated animals compared to their vehicle treated counterparts (FIG. 6A). Neither PTH nor PTH-βarr treatment had a significant effect on the RANKL or OPG expression in the β-arrestin 2−/− mice.

### 2. Example 2

The anabolic effects of PTH-(1-34) stimulation of the PTH1R on bone are also mediated by classic G protein-cAMP signaling, as well as a distinct mechanism independent of G protein recruitment, mediated by β-arrestin were demonstrated. Additionally, the bone resorptive effects of PTH1R stimulation appear to be predominantly G protein dependent mechanisms and not β-arrestin dependent.

Ligands capable of selectively stimulating G protein-independent/β-arrestin-dependent 7TMR signaling to ERK1/2 have also been described in the AT1A angiotensin receptor system using a synthetic angiotensin agonist peptide, [Sar₁,Ile₄,Ile₈]SII. Moreover, ligands originally classified as antagonists such as cardvedilol and inverse agonists ICI118551, for the β2-adrenergic receptor, and SR121463B for the V₂ vasopressin receptor have also been shown to promote scaffold assembly and β-arrestin-mediated MAPK activation. These observations indicate that β-arrestin recruitment is not exclusive to 7TMR G protein activation. The data presented here demonstrate biased agonism for PTH1R, where PTH-βarr can inhibit G protein-dependent signaling while activating -arrestin-dependent signaling ERK1/2 phosphorylayion in osteoblasts.

However, using a PTH1R ligand which preferentially activates β-arrestin mediated signaling while at the same time inhibits G protein recruitment conventional G protein signaling mechanisms are not sufficient to entirely account for the skeletal response of the β-arrestin −/− mice to PTH was demonstrated. Rather β-arrestin initiates a distinct signaling mechanism independent of G protein stimulation which contributes uniquely to the anabolic response in bone to PTH1R stimulation. Thus the attenuated response in bone anabolism reported in the β-arrestin−/− mice can in fact be due to the loss of this β-arrestin mediated signaling events rather than excess G protein signaling.

PTH1R stimulated G protein-mediated and G protein independent/β-arrestin-mediated mechanisms can differentially contribute to distinct elements of bone metabolism. β-arrestin mediated signaling events are indicated to be directed primarily at anabolic bone formation in trabecular bone, specifically increasing trabecular number and thickness, while not contributing to the bone resorptive effects of PTH1R stimulation.

A biased agonist, PTH-βarr, for the PTH1R that has the ability to selectively activate β-arrestin mediated signaling independent of G-protein activation that has a unique physiologic profile is disclosed herein. Moreover, compounds could also be biased in the opposite direction from PTH-barr that is preferentially activating G protein-mediated pathways while simultaneously antagonizing β-arrestin-dependent signaling pathways.

### L. REFERENCES

Ahn S, Shenoy S K, Wei H, Lefkowitz R J. (2004) Differential kinetic and spatial patterns of beta-arrestin and G protein-mediated ERK activation by the angiotensin II receptor. J. Biol. Chem. 279: 35518-35525.
Angers S, Salahpour A, Bouvier M. (2002) Dimerization: An emerging concept for G protein-coupled receptor ontogeny and function. Ann. Rev. Pharmacol. Toxicol. 42: 409-435.
Attramandal H, Arriza J L, Aoki C, Dawson T M, Codina J, Kwatra M M, Snyder S H, Caron M G, Lefkowitz R J. (1992) Beta\tilde{-}restin 2, a novel member of the arrestin/\tilde{b}eta-arrestin gene family. J. Biol. Chem. 267: 17882-17890.
Azzi M, Charest P G, Angers S, Rousseau G, Kohout T, Bouvier M, Pineyro G. (2003) Beta-arrestinmediated activation of MAPK by inverse agonists reveals distinct active conformations for G proteincoupled receptors. Proc. Natl. Acad. Sci. U.S.A. 100: 11406-11411.
Barlic J, Andrews J D, Kelvin A A, Bosinger S E, DeVries M E, Xu L, Dobransky T, Feldman R D, Ferguson S S G, Kelvin D J. (2000) Regulation of tyrosine kinase activation and granule release through β-arrestin by CXCRI. Nature Immunol. 1: 227-233.
Berg, K. A., Maayani, S., Goldfarb, J., Scaramellini, C., Leff, P., Clarke, W. P. (1998), Effector pathway-dependent relative efficacy at serotonin type 2A and 2C receptors: evidence for agonist-directed trafficking of receptor stimulus. Mol Pharmacol 54:94-104.
Bisello, A., Chorev, M., Rosenblatt, M., Monticelli, L., Mierke, D. F., Ferrari, S. L. (2002). Selective ligand-induced stabilization of active and desensitized parathyroid hormone type 1 receptor conformations. J Biol Chem 277:38524-38530.
Bockaert J, Marin P, Dumuis A, Fagni L. (2003) The ‘magic tail’ of G protein-coupled receptors: An anchorage for functional protein networks. FEBS Lett. 546: 65-72.
Brady A E, Limbird L E. (2002) G protein-coupled receptor interacting proteins: Emerging roles in localization and signal transduction. Cell. Signal. 14: 297-309.
Caunt, C. J., Finch, A. R., Sedgley, K. R., Oakley, L., Luttrell, L. M. and C. A., McArdle. Arrestin-mediated ERK activation by gonadotropin-releasing hormone receptors (GnRHRs): Receptor-specific activation mechanisms and compartmentalization. J. Biol. Chem., 281:2701-2710, 2006.
Craft C M, Whitmore D H, Weichmann A F. (1994) Cone arrestin identified by targeting expression of a functional family. J. Biol. Chem. 269: 4613-4619.
Daub H, Wallash C, Lankenau A, Herrlich A, Ullrich A. (1997) Signal characteristics of G proteintransactivated EGFR. EMBO J. 16: 7032-7044.
DeFea K A, Vaughn Z D, O'Bryan E M, Nishijima D, Dery O, Bunnett N R. (2000a) The proliferative and antiapoptotic effects of substance P are facilitated by formation of a beta-arrestin-dependent scaffolding complex. Proc. Natl. Acad. Sci. USA. 97:11086-11091.
DeFea K A, Zalevsky J, Thoma M S, Dery O, Mullins R D, Bunnett N R. (2000b) beta-Arrestin-dependent endocytosis of proteinase-activated receptor 2 is required for intracellular targeting of activated ERK1/2. J. Cell Biol. 148: 1267-1281.
Devi L. (2001) Heterodimerization of G-protein-coupled receptors: Pharmacology, signaling and trafficking. Trends Pharmacol. Sci. 22: 532-537.
Dikic I, Tokiwa G, Lev S, Courtneidge S A, Schlessinger J. (1996) A role for Pyk2 and Src in linking G protein-coupled receptors with MAP kinase activation. Nature. 383 547-550.
El Far O and Betz H. (2002) G-protein-coupled receptors for neurotransmitter amino acids: C-terminal tails, crowded signalosomes. Biochem J 365:329-336.
Elion E A. (2001) The STE5p scaffold. J Cell Sci. 114: 3967-3978.
Ferguson S S. (2001) Evolving concepts in G protein-coupled receptor endocytosis: The role in receptor desensitization and signaling. Pharm Rev. 53: 1-24. Flower D R. (1999) Modeling G-protein-coupled receptors for drug design. Biochim. Biophys. Acta. 1422:207-234.
Fredriksson, R., Lagerstrom, M. C., Lundin, L. G., and Schioth, H. B. (2003) The G-protein-coupled receptors in the human genome form five main families. Phylogenetic analysis, paralogon groups, and fingerprints. Mol. Pharmacol. 63, 1256-1272.
Freedman N J, Lefkowitz R J. (1996) Desensitization of G protein-coupled receptors. Recent Prog Horm Res. 51: 319-351.
Foord S M, Marshall F H. (1999) RAMPs: accessory proteins for seven transmembrane domain receptors. Trends Pharmacol. Sci. 20: 184-187.
Gardella, T. J., Luck, M. D., Jensen, G. S., Schipani, E., Potts, J. T. Jr., Juppner, H. (1996) Inverse agonism of amino-terminally truncated parathyroid hormone (PTH) and PTH-related peptide (PTHrP) analogs revealed with constitutively active mutant PTH/PTHrP receptor. Endocrinology 137:3936-3941.
Gensure R C, Gardella T J, Jüppner H. Parathyroid hormone and parathyroid hormone-related peptide, and their receptors. Biochem Biophys Res Commun. 328:666-678, 2005.
Gesty-Palmer D, El Shewy H, Kohout T A, Luttrell L M. (2005) beta-Arrestin 2 expression determines the transcriptional response to lysophosphatidic acid stimulation in murine embryo fibroblasts. J. Biol. Chem. 280: 32157-67.
Gesty-Palmer, Cen, M., Oakley, R., D., Reiter, E., Ahn, S., Nelson, C. D., Spurney, R. F., Luttrell, L. M., and R. J. Lefkowitz. (2006) Distinct conformations of the parathyroid hormone receptor mediate G protein and beta-arrestin dependent activation of ERK1/2. J. Biol. Chem. 281:10856-10864.
Ghanouni, P., Gryczynski, Z., Steenhuis, J. J., Lee, T. W., Farrens, D. L., Lakowicz, J. R. Kobilka, B. K. (2001). Functionally different agonists induce distinct conformations in the G protein coupling domain of the β2 adrenergic receptor. J Biol Chem 276:24433-24436.
Grewal S S, Fass D M, Yao H, Ellig C L, Goodman R H, Stork P J. (2000) Calcium and cAMP signals differentially regulate cAMP-responsive element-binding protein function via a Rap1-extracellular signal regulated kinase pathway. J. Biol. Chem. 275: 34433-34441.
Gurevich V V, Pals-Rylaarsdam R, Benovic J L, Hosey M M, Onorato J J. (1997) Agonist-receptor-arrestin, an alternative ternary complex with high agonist affinity. J. Biol. Chem. 272: 28849-28852.
Gutkind J S. (1998) The pathways connecting G protein-coupled receptors to the nucleus through divergent mitogen-activated protein kinase cascades. J Biol. Chem. 273: 1839-1842.
Hall R A, Lefkowitz R J. (2002) Regulation of G protein-coupled receptor signaling by scaffold proteins. Circ Res. 91: 672-680.
Hawes B E, van Biesen T, Koch W J, Luttrell L M, Lefkowitz R J. (1995) Distinct pathways of Gi- and Gqmediated mitogen-activated protein kinase activation. J. Biol. Chem. 270: 17148-17153.
Heeneman S, Haendeler J, Saito Y, Ishida M, Berk B C. (2000) Angiotensin I I induces transactivation of two different populations of the platelet-derived growth factor beta receptor. Key role for the p66 adaptor protein Shc. J. Biol. Chem. 275: 15926-15932.
Holst B, Hastrup H, Raffetseder U, Martini L, Schwartz T W. (2001) Two active molecular phenotypes of the tachykinin NK1 receptor revealed by G-protein fusions and mutagenesis. J. Biol. Chem. 276: 19793-19799.
Jafri, F., El Shewy, H., Luttrell, D. K., and L. M. Luttrell. Expression of a Chimeric Neurokinin NK-1 Receptor-β-Arrestin 1 Fusion Protein Produces Constitutive ERK1/2 Activation in HEK-293 Cells: Probing the Composition and Function of the G Protein-Coupled Receptor Signalsome'. J. Biol. Chem. 281:19346-19357, 2006.
Kenakin T (1995a) Agonist-receptor efficacy. I: Mechanisms of efficacy and receptor promiscuity. Trends Pharmacol Sci 16:188-192.
Kenakin T (1995b) Agonist-receptor efficacy. II. Agonist trafficking of receptor signals. Trends Pharmacol Sci 16:232-238.
Kenakin, T. (2002). Drug efficacy at G protein-coupled receptors. Annu Rev Pharmacol Toxicol 42:349-379.
Kolakowski, L. F. Jr. (1994) GCRDb: A G-protein coupled receptor database. Recept. Channels. 2, 1-7.
Krupnick J G, Goodman O B Jr, Keen J H, Benovic J L. Arrestin/clathrin interaction. Localization of the clathrin binding domain of nonvisual arrestins to the carboxy terminus. J Biol. Chem. 272:15011-6, 1997.
Lander E S, Linton L M, Birren B, Nusbaum C, Zody M C, Baldwin J, Devon K, Dewar K, Doyle M, FitzHugh W, et al. (2001) Initial sequencing and analysis of the human genome. Nature. 409: 860-921.
Laporte S A, Oakley R H, Zhang J, Holt J A, Ferguson S S, Caron M G, Barak L S. (1999) The beta2 adrenergic receptor/betaarrestin complex recruits the clathrin adaptor AP-2 during endocytosis. Proc Natl Acad Sci USA. 96:3712-3717.
Lefkowitz R J. (1993a) G protein-coupled receptor kinases. Cell 74: 409-412.
Lefkowitz, R. J., Cotecchia, S., Samama, P. Costa, T. (1993b). Constitutive activity of receptors coupled to guanine nucleotide regulatory proteins. Trends Pharmacol Sci 14:303-307.
Lev S, Moreno H, Martinez R, Canoll P, Peles E, Musacchio J M, Plowman G D, Rudy B, Schlessinger, J. (1995) Protein tyrosine kinase PYK2 involved in Ca(2+)-induced regulation of ion channel and MAP kinase functions. Nature. 376: 737-745.
Linseman D A, Benjamin C W, Jones D A. (1995) Convergence of angiotensin II and platelet-derived growth factor receptor signaling cascades in vascular smooth muscle cells. J. Biol. Chem. 270: 12563-12568.
Luttrell L M, Ferguson S S G, Daaka Y, Miller W E, Maudsley S, Della Rocca G J, Lin F-T, Kawakatsu H, Owada K, Luttrell D K, Caron M G, Lefkowitz R J. (1999a) beta-Arrestin-dependent formation of beta2 adrenergic receptor/Src protein kinase complexes. Science. 283: 655-661.
Luttrell, L. M., Daaka, Y., and R. J. Lefkowitz. (1999b) Regulation of tyrosine kinase cascades by G-protein coupled receptors. Curr Opin Cell Biol. 11: 177-183.
Luttrell L M, Roudabush F L, Choy E W, Miller W E, Field M E, Pierce K L, Lefkowitz R J. (2001) Activation and targeting of extracellular signal-regulated kinases by beta-arrestin scaffolds. Proc. Natl. Acad. Sci. USA. 98: 2449-2454.
Luttrell L M, Lefkowitz R J. (2002a) The role of beta-arrestins in the termination and transduction of G protein-coupled receptor signals. J. Cell. Sci. 115: 455-465.
Luttrell, L. M. Activation and targeting of MAP kinases by G protein-coupled receptors. (2002b) Can J Physiol Pharm. 80: 375-382.
Luttrell L M. (2005a) Regulators of GPCR activity: The Arrestins. In: Contemporary Clinical Neuroscience: The G Protein-Coupled Receptors Handbook, L. Devi, editor. Humana Press, Inc., Totowa, N.J. 159-198.
Luttrell L M. (2005b) Composition and function of G protein-coupled receptor ‘signalsomes’ controlling mitogen-activated protein kinase activity. J. Mol. Neurosci. 26: 253-264.
Luttrell L M. (2006) Transmembrane signaling by G protein-coupled receptors. Meth Mol. Biol. 332: 3-49.
Lohse M J, Benovic J L, Codina J, Caron M G, Lefkowitz R J. (1990) Beta-arrestin: A protein that regulates beta-adrenergic receptor function. Science. 248: 1547-1550.
Maudsley S, Martin B, Luttrell L M. (2005) Perspectives in Pharmacology: The origins of diversity and specificity in G protein-coupled receptor signaling. J. Pharm. Exp. Therapeutics. 314: 485-494.
McDonald P H, Chow C-W, Miller W E, LaPorte S A, Field M E, Lin F-T, Davis R J, Lefkowitz R J. (2000) beta-Arrestin 2: A receptor-regulated MAPK scaffold for the activation of JNK3. Science. 290: 1574-1577.
Meller, E., Puza, T., Diamond, J., Lieu, H. D. Bohmaker. K. (1992). Comparative effects of receptor inactivation, 17 β-estradiol and pertussis toxin on dopaminergic inhibition of prolactin secretion in vitro. J Pharmacol Exp Ther 263:462-469.
Miller W E, Lefkowitz R J. (2001) Expanding roles for beta-arrestins as scaffolds and adapters in GPCR signaling and trafficking. Curr. Opin. Cell Biol. 13: 139-145.
Milligan G. (2001) Oligomerisation of G-protein-coupled receptors. J. Cell Sci. 114: 1265-1271.
Murakami A, Yajima T, Sakuma H, McClaren M J, Inana G. (1993) X-arrestin: A new retinal arrestin mapping to the X chromosome. FEBS Lett. 334: 203-209.
Oakley R H, Laporte S A, Holt J A, Barak L S, Caron M G. (1999) Association of beta-arrestin with G proteincoupled receptors during clathrin-mediated endocytosis dictates the profile of receptor resensitization. J. Biol. Chem. 274: 32248-32257.
Pearson G, Robinson F, Beers Gibson T, Xu B E, Karandikar M, Berman K, Cobb M H. (2001) Mitogenactivated protein (MAP) kinase pathways: regulation and physiological functions. Endocr Rev. 22:153-183.
Perez, D. M., Hwa, J., Gaivin, R., Mathur, M., Brown, F., Graham, R. M. (1996). Constitutive activation of a single effector pathway: evidence for multiple activation states of a G protein-coupled receptor. Mol Pharmacol 49:112-122.
Perry S J, Lefkowitz R J. (2002a) Arresting developments in heptahelical receptor signaling and regulation. Trends Cell Biol. 12: 130-138.
Perry S J, Baillie G S, Kohout T A, McPhee I, Magiera M M, Ang K L, Miller W E, McLean A J, Conti M, Houslay M D, Lefkowitz R J. (2002b) Targeting of cyclic AMP degradation to beta 2-adrenergic receptors by beta-arrestins. Science 298: 834-836.
Potts J T. Parathyroid hormone: past and present. J Endocrinol. 187:311-25, 2005
Prenzel N, Zwick E, Daub H, Leserer M, Abraham R, Wallasch C, Ullrich A. (1999) EGFR transactivation by G-protein-coupled receptors requires metalloproteinase cleavage of proHB-EGF. Nature. 402: 884-888.
Sagan, S., Karoyan, P., Chassaing, G., Lavielle, S. (1999). Further delineation of the two binding sites (R*(n)) associated with tachykinin neurokinin-1 receptors using [3-Prolinomethionine(11)]SP analogues. J Biol Chem 274:23770-23776.
Samama, P., Cotecchia, S., Costa, T., Lefkowitz, R. J. (1993). A mutation-induced activated state of the β2-adrenergic receptor. Extending the ternary complex model. J Biol Chem 268:4625-4636.
Seifert, R., Gether, U., Wenzel-Seifert, K., Kobilka, B. K. (1999). Effects of guanine, inosine, and xanthine nucleotides on β(2)-adrenergic receptor/G(s) interactions: evidence for multiple receptor conformations. Mol Pharmacol 56:348-358.
Sexton P M, Albiston A, Morfis M, Tilakaratne N. (2001) Receptor activity modifying proteins. Cell. Signal. 13: 73-83.
Shenoy S K, McDonald P H, Kohout T A, Lefkowitz R J. (2001) Regulation of receptor fate by ubiquitination of activated beta2-adrenergic receptor and beta-arrestin. Science 294: 1307-1313.
Shenoy S K, Lefkowitz R J (2003) Multifaceted roles of beta-arrestins in the regulation of seven-membranespanning receptor trafficking and signalling. Biochem. 1 375: 503-515.
Shenoy S K, Lefkowitz R J. (2005) Seven-transmembrane receptor signaling through beta-arrestin. Sci STKE. 2005(308):cm10.
Shenoy S K, Lefkowitz R J. (2005) Angiotensin II-stimulated signaling through G proteins and beta-arrestin. Sci STKE. 2005(311):cm14.
Shenoy S K, Lefkowitz R J. (2005) Receptor regulation: beta-arrestin moves up a notch. Nat Cell Biol. 7:1159-1161.
Shenoy S K, Drake M T, Nelson C D, Houtz D A, Xiao K, Madabushi S, Reiter E, Premont R T, Lichtarge O, Lefkowitz R J. (2006) beta-arrestin-dependent, G protein-independent ERK1/2 activation by the beta2 adrenergic receptor. J Biol. Chem. 281: 1261-1273.
Shinohara T, Dietzschold B, Craft C M, Wistow G, Early J J, Donoso L A, Horwitz J, Tao R. Primary and secondary structure of bovine retinal S antigen (48-kDa protein). (1987) Proc. Nat. Acad. Sci., USA. 84: 6975-6979.
Sneddon W B, Magyar C E, Willick G E, Syme C A, Galbiati F, Bisello A, Friedman P A. (2004). Ligand-selective dissociation of activation and internalization of the parathyroid hormone (PTH) receptor: conditional efficacy of PTH peptide fragments. Endocrinology 145:2815-2823.
Spengler, D., Waeber, C., Pantaloni, C., Holsboer, F., Bockaert, J., Seeburg, P. H., Journot, L. (1993). Differential signal transduction by five splice variants of the PACAP receptor. Nature 365:170-175.
Tohgo A, Pierce K L, Choy E W, Lefkowitz R J, Luttrell L M. (2002) Beta-Arrestin scaffolding of the ERK cascade enhances cytosolic ERK activity but inhibits ERK-mediated transcription following angiotensin AT1a receptor stimulation. J. Biol. Chem. 277: 9429-9436.
Tohgo A, Choy E W, Gesty-Palmer D, Pierce K L, Laporte S, Oakley R H, Caron M G, Lefkowitz R J, Luttrell L M. (2003) The stability of the G protein-coupled receptor-beta-arrestin interaction determines the mechanism and functional consequence of ERK activation. J. Biol. Chem. 278: 6258-6267.
van Biesen T, Luttrell L M, Hawes B E, Lefkowitz R J. (1996) Mitogenic signaling via G protein-coupled receptors. Endocr Rev 17: 698-714.
Venter J C, Adams M D, Myers E W, Li P W, Mural R J, Sutton G G, Smith H O, Yandell M, Evans C A, Holt R A, et al. (2001) The sequence of the human genome. Science. 291: 1304-1351.
Vossler M R, Yao H, York R D, Pan M G, Rim C S, Stork P J. (1997) cAMP activates MAP kinase and Elk-1 through a B-Raf- and Rap1-dependent pathway. Cell 89: 73-82.
Wade S M, Lim W K, Lan K L, Chung D A, Nanamori M and Neubig R R (1999) G(i) activator region of α(2A)-adrenergic receptors: distinct basic residues mediate G(i) versus G(s) activation. Mol Pharmacol 56:1005-1013.
Wei, H., Ahn, S., Shenoy, S. K., Karnik, S., Hunyady, L., Luttrell, L. M., and R. J. Lefkowitz. Independent G protein and β-Arrestin2 mediated activation of ERK by angiotensin. Proc. Natl. Acad. Sci., U.S.A., 100:10782-10787, 2003.
Whitfield J F. Osteoporosis-treating parathyroid hormone peptides: What are they? What do they do? How might they do it? Curr Opin Investig Drugs. 7:349-59, 2006.
Yamaki K, Takahashi Y, Sakuragi S, Matsubara K. (1987) Molecular cloning of the S-antigen cDNA from bovine retina. Biochem. Biophys. Res. Commun. 142: 904-910.
Zhang J, Ferguson S S, Barak L S, Menard L, Caron M G. (1996) Dynamin and beta-arrestin reveal distinct mechanisms for G protein-coupled receptor internalization. J. Biol. Chem. 271: 18302-18305.

M. Sequences

| 1. SEQ ID NO: 1 TYPE 1 PARATHYROID HORMONE RECEPTOR | |
|---|---|
| [Homo sapiens] ACCESSION AAI10389 REFERENCE: Strausberg RL, et | |
| al. Generation and initial analysis of more than 15,000 full-length human | |
| and mouse cDNA sequences Proc. Natl. Acad. Sci. U.S.A. 99: 16899-16903, | |
| 2002. AMINO ACID SEQUENCE (436 amino acids): | |
| Origin | |
| 1 | mgtariapgl alllccpvls sayalvdadd vmtkeeqifl lhraqaqcgk rlkevlqrpa |
| 61 | simesdkgwt sastsgkprk dkasgklype seedkeaptg sryrgrpclp ewdhilcwpl |
| 121 | gapgevvavp cpdyiydfnh kghayrrcdr ngswelvpgh nrtwanysec vkfltnetre |
| 181 | revfdrlgmi ytvgysvsla sltvavlila yfrrlhctrn yihmhlflsf mlravsifvk |
| 241 | davlysgatl deaerlteee lraiaqappp pataaagyag crvavtffly flatnyywil |
| 301 | veglylhsli fmaffsekky lwgftvfgwg lpavfvavwv svratlantg cwdlssgnkk |
| 361 | wiiqvpilas ivlnfilfin ivrvlatklr etnagrcdtr qqyrkllkst lvlmplfgvh |
| 421 | yivfmatpyt evsgtlwqvq mhyemlfnsf qgffvaiiyc fcngevqaei kkswsrwtla |
| 481 | ldfkrkarsg sssysygpmv shtsvtnvgp rvglglplsp rllptattng hpqlpghakp |
| 541 | gtpaletlet tppamaapkd dgflngscsg ldeeasgper ppallqeewe tvm |
| 2. Human PTH(1-84) Accession AAH96144 REFERENCE: Strausberg | |
| RL, et al. Generation and initial analysis of more than 15,000 full-length | |
| human and mouse cDNA sequences Proc. Natl. Acad. Sci. U.S.A. 99: 16899- | |
| 16903, 2002. | |
| Origin | |
| 1 | mipakdmakv mivmlaicfl tksdgksvkk rsyseiqlmh nlgkhlnsme rvewlrkklq |
| 61 | dvhnfvalga plaprdagsq rprkkednvl veshekslge adkadvnvlt kaksq |
| 3. Human PTHrP precursor (Contains PTHrP[1-36], PTHrP[38-84; and | |
| osteostatin, which are generated by proteolysis) Accession P12272, | |
| REFERENCE: Gerhart DS, et al. The status, quality, and expansion of the | |
| NIH full-length cDNA project: the Mammalian Gene Collection (MGC). | |
| Genome Res. 14: 2121-2127, 2004. | |
| Origin | |
| 1 | mqrrlvqqws vavfllsyav pscgrsvegl srrlkrayse hqllhdkgks iqdlrrrffl |
| 61 | hhliaeihta eiratsevsp nskpspntkn hpvrfgsdde gryltqetnk vetykeqplk |
| 121 | tpgkkkkgkp gkrkeqekkk rrtrsawlds gvtgsglegd hlsdtsttsl eldsrrh |
| 4. SEQ ID NO: 4 TYPE 1 PARATHYROID HORMONE RECEPTOR | |
| [Homo sapiens] mRNA ACCESSION BC110388. | |
| 1 | atggggaccg cccggatcgc acccggcctg gcgctcctgc tctgctgccc cgtgctcagc |
| 61 | tccgcgtacg cgctggtgga tgcagatgac gtcatgacta aagaggaaca gatcttcctg |
| 121 | ctgcaccgtg ctcaggccca gtgcggaaaa cggctcaagg aggtcctgca gaggccagcc |
| 181 | agcataatgg aatcagacaa gggatggaca tctgcgtcca catcagggaa gcccaggaaa |
| 241 | gataaggcat ctgggaagct ctaccctgag tctgaggagg acaaggaggc acccactggc |
| 301 | agcaggtacc gagggcgccc ctgtctgccg gaatgggacc acatcctgtg ctggccgctg |
| 361 | ggggcaccag gtgaggtggt ggctgtgccc tgtccggact acatttatga cttcaatcac |
| 421 | aaaggccatg cctaccgacg ctgtgaccgc aatggcagct gggagctggt gcctgggcac |
| 481 | aacaggacgt gggccaacta cagcgagtgt gtcaaatttc tcaccaatga gactcgtgaa |
| 541 | cgggaggtgt ttgaccgcct gggcatgatt tacaccgtgg gctactccgt gtccctggcg |
| 601 | tccctcaccg tagctgtgct catcctggcc tactttaggc ggctgcactg cacgcgcaac |
| 661 | tacatccaca tgcacctgtt cctgtccttc atgctgcgcg ccgtgagcat cttcgtcaag |
| 721 | gacgctgtgc tctactctgg cgccacgctt gatgaggctg agcgcctcac cgaggaggag |
| 781 | ctgcgcgcca tcgcccaggc gcccccgccg cctgccaccg ccgctgccgg ctacgcgggc |
| 841 | tgcagggtgg ctgtgacctt cttcctttac ttcctggcca ccaactacta ctggattctg |
| 901 | gtggaggggc tgtacctgca cagcctcatc ttcatggcct tcttctcaga gaagaagtac |
| 961 | ctgtggggct tcacagtctt cggctggggt ctgcccgctg tcttcgtggc tgtgtgggtc |
| 1021 | agtgtcagag ctaccctggc caacaccggg tgctgggact tgagctccgg gaacaaaaag |
| 1081 | tggatcatcc aggtgcccat cctggcctcc attgtgctca acttcatcct cttcatcaat |
| 1141 | atcgtccggg tgctcgccac caagctgcgg gagaccaacg ccggccggtg tgacacacgg |
| 1201 | cagcagtacc ggaagctgct caaatccacg ctggtgctca tgcccctctt tggcgtccac |
| 1261 | tacattgtct tcatggccac accatacacc gaggtctcag ggacgctctg gcaagtccag |
| 1321 | atgcactatg agatgctctt caactccttc cagggatttt ttgtcgcaat catatactgt |
| 1381 | ttctgcaacg gcgaggtaca agctgagatc aagaaatctt ggagccgctg gacactggca |
| 1441 | ctggacttca agcgaaaggc acgcagcggg agcagcagct atagctacgg ccccatggtg |
| 1501 | tcccacacaa gtgtgaccaa tgtcggcccc cgtgtgggac tcggcctgcc cctcagcccc |
| 1561 | cgcctactgc ccactgccac caccaacggc caccctcagc tgcctggcca tgccaagcca |
| 1621 | gggaccccag ccctggagac cctcgagacc acaccacctg ccatggctgc tcccaaggac |
| 1681 | gatgggttcc tcaacggctc ctgctcaggc ctggacgagg aggcctctgg gcctgagcgg |
| 1741 | ccacctgccc tgctacagga agagtgggag acagtcatgt ga |
| 5. SEQ ID NO: 5 Human PTH Accession BC096144 | |
| 1 | atgatacctg caaaagacat ggctaaagtt atgattgtca tgttggcaat ttgttttctt |
| 61 | acaaaatcgg atgggaaatc tgttaagaag agatctgtga gtgaaataca gcttatgcat |
| 121 | aacctgggaa aacatctgaa ctcgatggag agagtagaat ggctgcgtaa gaagctgcag |
| 181 | gatgtgcaca attttgttgc ccttggagct cctctagctc ccagagatgc tggttcccag |
| 241 | aggccccgaa aaaaggaaga caatgtcttg gttgagagcc atgaaaaaag tcttggagag |
| 301 | gcagacaaag ctgatgtgaa tgtattaact aaagctaaat cccagtga |

1.-20. (canceled)

21. A method of promoting anabolic bone growth in a subject, the method comprising administering to the subject a biased ligand for the parathyroid hormone (PTH)/PTH-related protein receptor (PTH1R) in an amount sufficient to effect promotion of anabolic bone growth.

22. The method of claim 21, wherein the biased ligand stimulates β-arrestin-mediated ERK1/2 signaling independent of G protein-mediated signaling.

23. The method of claim 21, wherein the biased ligand is (D-Trp12,Tyr34)PTH(7-34).

24. The method of claim 21, wherein the biased ligand increases trabecular bone formation.

25. The method of claim 21, further comprising monitoring the modulation of the PTH1R by analyzing a biofluid of the subject for at least one marker indicating biased ligand modulation.

26. The method of claim 25, wherein the biofluid is selected from urine and serum.

27. The method of claim 25, wherein at least one marker is selected from osteocalcin and deoxypyridinoline.

28. A method of treating a bone disorder, the method comprising administering to a subject in need thereof a biased ligand for the PTH1 receptor, in an amount sufficient to treat the bone disorder.

29. The method of claim 28, wherein the bone disorder is selected from osteoporosis and osteopenia.

30. The method of claim 28, wherein the biased ligand stimulates β-arrestin-mediated signaling independent of G protein-mediated signaling.

31. The method of claim 28, wherein the biased ligand is (D-Trp12,Tyr34)PTH(7-34).

32. The method of claim 28, further comprising monitoring the modulation of the PTH1R by analyzing a biofluid of the subject for at least one marker indicating biased ligand modulation.

33. The method of claim 32, wherein the biofluid is selected from urine and serum.

34. The method of claim 32, wherein at least one marker is selected from osteocalcin and deoxypyridinoline.

35. A method of treating hyperparathyroidism, the method comprising administering to a subject in need thereof a biased ligand for the PTH1 receptor, in an amount sufficient to treat hyperparathyroidism.

36. The method of claim 35, wherein the biased ligand stimulates β-arrestin-mediated signaling independent of G protein-mediated signaling.

37. The method of claim 35, wherein the biased ligand is (D-Trp12,Tyr34)PTH(7-34).

38. The method of claim 35, further comprising monitoring the modulation of the PTH1R by analyzing a biofluid of the subject for at least one marker indicating biased ligand modulation.

39. The method of claim 38, wherein the biofluid is selected from urine and serum.

40. The method of claim 38, wherein at least one marker is selected from osteocalcin and deoxypyridinoline.

41. The method of claim 21, wherein the biased ligand is continuously administered.

42. The method of claim 41, wherein the biased ligand is continuously administered in an amount and under conditions such that trabecular bone volume or trabecular bone number are increased in the subject without increasing the rate of bone resorption.

43. The method of claim 41, wherein the subject is a human.

44. The method of claim 41, wherein the bone disorder is a metabolic bone disease.

45. The method of claim 28, wherein the biased ligand is continuously administered.

46. The method of claim 45, wherein the biased ligand is continuously administered in an amount and under conditions such that trabecular bone volume or trabecular bone number are increased in the subject without increasing the rate of bone resorption.

47. The method of claim 45, wherein the subject is a human.

48. The method of claim 45, wherein the bone disorder is a metabolic bone disease.
